(19) United States
(12) Patent Application Publication
(10) Pub. No.: US 2008/0094573 A1
(43) Pub. Date: Apr. 24, 2008

(54) SURFACE-MODIFIED MATERIALS, SUCH AS CONTACT LENSES, METHODS AND KITS FOR THEIR PREPARATION, AND USES THEREOF

(76) Inventors: Patrick VERMETTE, Sherbrooke (CA); Anne Danion, Venisseux (FR)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/695,892

(22) Filed: Apr. 3, 2007

Related U.S. Application Data
(60) Provisional application No. 60/788,700, filed on Apr. 4, 2006.

(51) Int. Cl. G02C 7/04; A61K 9/00; A61K 31/5383; A61P 27/02

(52) U.S. Cl. 351/177; 424/429; 514/230.2; 424/9.1

(57) ABSTRACT

Modified contact lenses and surfaces, methods for their preparation, and corresponding kits are disclosed. Also disclosed are uses of the contact lenses, such as for the administration of an agent to the eye.

### CROSS REFERENCE TO RELATED APPLICATIONS

This application claims the benefit, under 35 U.S.C. § 119(e), of U.S. provisional application Ser. No. 60/788,700 filed on Apr. 4, 2006, which is incorporated herein by reference in its entirety.

### FIELD OF THE INVENTION

The present invention relates to methods of surface modification, such as the surface of a contact lens, surface-modified products, such as contact lenses, produced by these methods, and their uses.

### BACKGROUND OF THE INVENTION

Currently 50 million people in the world are blind and 150 million have some degree of visual impairment. In 1990, the aggregated cost of blindness to the US federal budget was estimated to be approximately US$ 4.1 billion. More importantly, it has been estimated that in the USA, if all the avoidable blindness in persons under 20 and working-age adults were prevented, a potential saving of US$ 1 billion per year would accrue to the federal budget. In addition to being a public health problem, blindness and visual impairment have important socioeconomic implications.

As an example, glaucoma, a familial blinding disease, affects up 3 to 5 percent of the population. Glaucoma is considered to be the third largest cause of blindness worldwide after cataract and trachoma, and is responsible for an estimated 5.2 million cases. Estimates prepared by WHO put the total number of suspect cases of glaucoma at around 105 million. In glaucoma the increase of the intra-ocular pressure progressively damages the optic nerve leading to blindness if left untreated. Treatment involves different eye-drops one to many times a day for a lifetime. The molecules are topical beta-blockers (Timolol, Levobunolol, Betaxolol, etc.), Apraclonidine, Pilocarpine, Dorzolamide, Brinzolamide, etc. One of the main problems in treating glaucoma is compliance: the patients forget or neglect to put their drops, because they do not feel sick and because of the frequency and inconvenience of the drug administration, thus allowing the disease to progress.

Another widespread eye disease, corneal ulcers, can arise from microbes of different origins. In most of the cases, more than one antibiotic has to be used on a frequent schedule. Many ocular inflammations such as chronic uveitis or chronic keratitis (immune or post-viral diseases) require the long-term use of anti-inflammatory agents.

In the case of corneal graft, no systemic drugs are normally used to prevent rejection, but patients are put on long-term topical corticosteroids for years with the potential side effects of cataract formation and secondary glaucoma.

Defects in the tear film, chemical or foreign body trauma, allergic hypersensitivity reactions, and overuse of contact lenses, as well as complications after laser in situ keratomileusis, can result in injury to the ocular surface and predispose the cornea to infection. (Garg P et al. 2001 Ophthalmology 108:121-125; Liesegang T J. 1988, In Kaufman H E, Barron B A, McDonald M B, Waltman S R, editors. The cornea. New York: Churchill Livingstone. p 217-270.)

Because of its high incidence and potential complications, bacterial keratitis is one of the most threatening ocular infections. Pseudomonas aeruginosa and Staphylococcus aureus frequently cause severe keratitis that may lead to progressive destruction of the corneal epithelium and stroma. (Alexandrakis G, et al. 2000 Ophthalmology 107:1497-1502; Bourcier T et al. 2003. Br J Opthalmol 87:834-838.)

Infectious keratitis due to these organisms often causes corneal scarring, corneal perforation, and blindness if aggressive and appropriate therapy is not promptly initiated. (Callegan M C, et al. 1994, Clin Pharmacokinet 27:129-149; Holland S P, et al. 1993. In: Fick R B, editor. Pseudomonas aeruginosa: the opportunist. Boca Raton: CRC Press Inc. p 160-176.)

Successful therapy of bacterial keratitis must be able to rapidly attain high drug concentrations at the site of infection. Since the cornea is not vascularized, it is not readily permeated by systemically administered drugs, which are therefore generally not used for the treatment of keratitis. (Callegan M C, et al. 1994, supra) On the other hand, topical treatment may fail to achieve therapeutically active drug levels in the cornea, as continuous tear flow reduces the bioavailability of topically applied antibiotics and the corneal epithelium acts as a barrier against drug penetration.

For these reasons, standard treatment of severe bacterial keratitis requires administration at frequent intervals (every 15 to 60 min for 48 to 72 h) of eye-drops often containing fortified solutions of fluoroquinolones (more concentrated than commercially available solutions) or multiple antibiotics, usually a cephalosporin and an aminoglycoside. (Callegan M C, et al. 1994 supra) However, this regimen not only is disruptive to the patient and usually necessitates hospitalization, but it has also been associated with in vitro toxicity to the corneal epithelium.

Efforts are now directed to testing new antimicrobials that better permeate the cornea and developing delivery systems capable of prolonging the contact time between antibiotics and the corneal tissue, thereby potentially enhancing intra-corneal delivery of ophthalmic medication.

Pharmaceutical companies have exhausted the arsenal of known antibiotic classes. Currently, the launch of new antibacterial products usually includes stronger dose formulations of old antibiotic classes. This results in the development of more antibiotic resistant strains and increased resistance of current strains. New classes of antibiotics and delivery systems that would limit the use of massive concentration of antibiotics are needed. Two major ocular pathogens that have demonstrated widespread antibiotic resistance are Staphylococcus aureus and Pseudomonas aeruginosa.

When a person suffers from eye ailments today, eye drops are prescribed nine times out of ten to treat the ocular disease and/or relieve discomfort. Despite the excellent acceptance by patients, one of the major problems encountered is rapid pre-corneal drug loss. In fact, 95% of the drug administered in this manner flows out of where it is needed. Upon application, eye drops usually mix with tears that are quickly drained into the nasal cavity, with subsequent passage into the blood stream increasing the risk of side effects. For example, the glaucoma drug Timolol can cause heart problems.

Colloidal systems have also been considered for ophthalmic applications, however, they also suffer from the problems noted above in that drug uptake is limited because the colloidal suspensions are quickly washed away by tearing action.

Furthermore, because of this above-noted rapid clearance, an ophthalmic drug has to be administered several times a day. The frequent doses reduce patient compliance and can be quite uncomfortable for the patient, as in the case of anti-glaucoma drugs which cause blurred vision for hours after application. In addition, dose concentration and regimen through eye drops are inconsistent and difficult to regulate, since the majority of the drug is released in an initial concentration burst.

Thus, there is a need for ophthalmic drug delivery systems that increase the residence time of the drug into the eye region, thereby reducing wastage and decreasing side effects. Several types of ophthalmic drug delivery systems have been proposed to provide localized or controlled sustained drug release over time including: hydrogels, cyclodextrins, collagen shields and contact lenses, used either alone or loaded with therapeutic agents, and colloidal systems suspended in a liquid or ointment carrier.

Most hydrogels offer only moderate to marginal improvement of ocular drug bioavailability and can cause blurred vision. Cyclodextrins are an alternative approach to increase the solubility of the drug in solution and to increase corneal permeability. However, they do not provide a sustained drug release. Collagen shields have been developed as a delivery system for drugs that need high and sustained levels to the cornea. However, collagen shields do not provide a sustained drug release into the eye as demonstrated by Phinney R B et al. (Phinney R B et al., Arch Ophtalmol. 1988; 106: 1599-1604).

Soft contact lenses have been described for the management of many ophthalmic disorders [G. Smollin, M. H. Friedlaender (eds) International Ophthalmology Clinics, vol. 31, 2, (1991)]. Contact lenses can be loaded with medications by pre-soaking them in a medication solution for therapeutic applications. However, these pre-soaked contact lenses provide a marginal means of delivery because therapeutics freely dispersed within the contact lens structure are rapidly released (i.e., burst-release), often leading to increased topical drug side effects and toxicity reactions [G. A. Lesher, G. G. Gunderson, Optom. Vis. Sci. 70 (1993), pp. 1012-1018]. Furthermore, many polymers from which the lenses are made cannot be loaded with diffusible drugs owing to insufficient solubility of the drug into the polymer or an inadequate diffusion rate of the drug through and out of the polymeric materials.

There is thus a continued need for improved systems for ophthalmic applications, such as a suitably modified contact lens. Further, there is a continued need for surface modification strategies, such as those which may be employed in the modification of contact lenses.

The present description refers to a number of documents, the content of which is herein incorporated by reference in their entirety.

### SUMMARY OF THE INVENTION

The invention relates to the modification of a product or surface, such as that of a contact lens, products or surfaces (such as contact lenses) so modified, uses thereof, and corresponding kits.

In an aspect, the present invention provides a method of modifying a contact lens with a carrier moiety capable of associating with an agent. This method comprises the step of permitting the binding of an adapter molecule to a first ligand attached to the contact lens and a second ligand attached to the carrier moiety. In embodiments, such binding may be effected in one or more steps, via the incubation of one or more of the contact lens comprising a first ligand attached thereto and the carrier moiety comprising a second ligand attached thereto, with the adapter molecule capable of concurrent binding to said first and second ligands, under conditions permitting binding of the adapter molecule to the first and second ligands, wherein the first and second ligands may be the same or different.

In embodiments, such incubations may be effected sequentially or concurrently.

The present invention also provides a kit for modifying a contact lens comprising a contact lens; a carrier moiety; and an adapter molecule capable of binding the first and second ligands.

In an embodiment, the kit may further comprise a second adaptor molecule capable of binding the second ligands. This kit may further comprise instructions setting forth any of the embodiments of the method of the invention.

The present invention also provides a contact lens whereto carrier moieties are bound, wherein both the first and the second ligands are concurrently bound to an adapter molecule.

In an embodiment, the contact lens comprises the first ligand, which is attached thereto, and the carrier moiety comprises the second ligand, which is attached thereto. Also, the adapter molecule is capable of concurrently binding the first and the second ligands. The first and second ligands may be the same or may be different.

In an embodiment, the contact lens may comprise a plurality of layers of carrier moieties attached thereto.

In an embodiment, the method comprises the steps of incubating the contact lens with the adapter molecule under conditions permitting the binding of the first ligand to the adapter molecule; and incubating this adapter-molecule modified contact lens with the carrier moiety under conditions permitting the binding of the second ligand to the adapter molecule.

In an embodiment, the method may further comprise the steps of incubating this carrier-modified contact lens, wherein the carrier moiety is a first carrier moiety, with a second adaptor molecule capable of binding a ligand attached to the first carrier moiety, under conditions permitting the binding of the second adaptor molecule to the first carrier-attached ligand; and incubating this second adapter molecule-modified contact lens with a second carrier moiety which may be the same or different from the first carrier moiety, the second carrier moiety comprising a ligand attached thereto capable of binding the second adapter molecule, under conditions permitting the binding of the second carrier-attached ligand to the second adapter molecule. These two steps may further be repeated one or more times with further adapter molecules and ligand-attached carrier moieties.

In a further embodiment, the carrier moiety comprising second ligands attached thereto may be in the form of aggregates of carrier moieties which are previously formed by incubating the carrier moieties with adapter molecules under conditions permitting the formation of aggregates via binding of the second ligands to the adapter molecules.

In another embodiment, the method may comprise the steps of incubating the carrier moiety with the adapter molecule under conditions permitting binding of the second ligand to the adapter molecule; and incubating this adapter-molecule modified carrier with the contact lens under conditions permitting binding of the first ligand to the adapter molecule.

In another embodiment, the method comprises the steps of incubating the contact lens with the carrier moiety and the adapter molecule, under conditions permitting binding of the first and second ligands to the adapter molecule.

In an embodiment, the adapter molecule is capable of homospecific binding and the first and second ligands are the same. In an alternative embodiment, the adapter molecule is capable of heterospecific binding and the first and second ligands are different.

In other embodiments, the first and second ligands may be the same and/or the first and second adapter molecules may be the same. Also, any of the ligands may be biotin and any of the adapter molecules may be a biotin-binding protein, such as a biotin-binding protein selected from the group consisting of avidin, NeutrAvidin, and streptavidin.

In embodiments, the carrier moieties may be liposomes.

In the method, kit and/or contact lens of the present invention the contact lens may have been incubated, prior to binding the adapter molecule to the first and second ligands, in a solution of a polymer under conditions permitting the reaction of a first functional group with a second functional group to form a covalent link

In an embodiment, the contact lens comprises the first functional group attached thereto, the polymer comprises the second functional group attached thereto, and the first and second functional groups can react with each other to form a covalent link. In an embodiment, the solution is in cloud point conditions.

The invention also provides a method of modifying a surface (of a material, article of manufacture or product) comprising incubating the surface in a solution of a polymer, in cloud point conditions under conditions permitting the reaction of a first functional group with a second functional group. The invention also relates to a surface or surface-modified material, article or product produced by this method.

The invention also provides a kit for modifying a surface comprising first functional groups attached thereto, the kit comprising a solution of a polymer in cloud point conditions and instructions for incubating the surface in the polymer solution.

The invention also provides an article of manufacture (e.g. a contact lens) comprising a surface whereto a polymer has been covalently linked by incubating the article or surface thereof in a solution of the polymer in cloud point conditions under conditions permitting the reaction of first and second functional groups. In an embodiment, the surface may be a surface of a contact lens.

In embodiments, the first functional group is an amine and the second functional group is an ester, the polymer is a PEG-based polymer and/or the polymer is NHS-PEG-Biotin. Also, the polymer may be low-fouling. More specifically, it may be carboxymethyl dextran.

In the method, kit and contact lens of the invention, the contact lens may comprise a third functional group attached thereto, which has been activated, prior to binding of the adapter molecule to the first and second ligands, by incubating the contact lens in the presence of a coupling agent capable of reacting with the third functional group and activate it.

The present invention also provides a method for modifying a contact lens which comprises third functional groups attached thereto, comprising incubating the contact lens in the presence of a coupling agent capable of reacting with the third functional groups and activating them.

The present invention also provides a kit for modifying a contact lens, which comprises third functional groups attached thereto. The kit may comprise a coupling agent capable of reacting with the third functional group to activate it, and instructions for incubating the contact lens in the presence of the coupling agent.

The present invention also provides a contact lens, which comprises third functional groups attached thereto, wherein the third functional groups have been activated by incubating the contact lens in the presence of a coupling agent capable of reacting with the third functional groups and activate them.

In an embodiment of the invention, the third functional group is a carboxyl or a hydroxyl group. In another embodiment, the third functional group is a hydroxyl functional group and the coupling agent is a hydroxyl-reactive coupling agent. In further embodiments, the coupling agents are N,N′-disuccinimidyl carbonate (DSC), epoxides, oxiranes, oxidation with periodate, enzymatic oxidation, alkyl halogens, isocyanates, or carbonyldiimidazole (CDI).

The present invention also provides a contact lens which is produced by any of the methods or embodiments of the invention.

In embodiments, the carrier moiety may comprise one or more agents. The agent may be selected from the group consisting of drugs, ophthalmic drugs, proteins, hormones, enzymes, amino acids, polypeptides, medicaments, therapeutics, vitamins, antibiotics (e.g., levofloxacin), anti-inflammatory agents and anti-rejection agents.

In an embodiment, the agent is encapsulated in the carrier moiety. In a further embodiment the agent is encapsulated in the carrier moiety and the modified contact lens moiety is also incubated in the agent during the preparation of the modified contact lens. As such, in an embodiment, the contact lens comprises carrier moieties bound thereto whereby the carrier moieties have the agent encapsulated therein, and the contact lens is also associated with agent that is not encapsulated within the carrier moieties.

In an embodiment, the contact lens of the invention may be used for the administration of the agent in the vicinity of an eye. This administration may comprise the controlled release of the agent.

In an embodiment, the contact lens of the invention may be used for improving wear comfort.

In embodiments, the contact lens of the invention may be used for treating a condition selected from the group consisting of ocular disease, ocular infection, ocular infectious diseases and ocular inflammation. The condition may be selected from the group consisting of chronic uveitis, ocular wound (i.e. to aid ocular wound healing), dry eye, chronic keratitis, glaucoma, corneal ulcer and contact lens wear discomfort.

In embodiments, the contact lens of the invention may be used for preventing the rejection of corneal graft. The contact lens of the invention may also be used for the preparation of a therapeutic device for administering an agent to the eye of a patient in need thereof.

The present invention also provides a method for administering an agent to the eye of a subject comprising placing the contact lens of the invention in contact with the eye of the subject.

In embodiments, the agent may be a sensor (e.g. a fluorescently labeled glucose receptor or competitor). In embodiments, the contact lens of the invention may be used as an in vivo analyte sensor (e.g. as an ocular glucose sensor).

The invention further provides a package comprising the above-noted contact lens together with instructions for administering an agent to or bringing an agent into contact with the eye.

In an embodiment the subject is a mammal, in a further embodiment a human.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 represents an example of the chemical reactions leading to the attachment of liposomes onto the surface of soft contact lenses;

FIG. 2 shows high-resolution XPS C is spectra recorded at various stages of fabrication of the multi-layer construct: cleaned contact lens surface (contact lens), contact lens plus grafted poly(ethylenimine) (PEI), contact lens+PEI+PEG-Biotin, contact lens+PEI+PEG-Biotin+NeutrAvidin (Neut), and one layer of liposomes docked onto the NeutrAvidin layer;

FIG. 3 shows results of ELISA assays of contact lenses modified with PEG layers produced without cloud point grafting conditions with a (−) negative control corresponding to a NeutrAvidin-coated lens deactivated with free biotin and a (+) positive control corresponding to an active NeutrAvidin-coated lens;

FIG. 4 shows results of ELISA assays of contact lenses modified with PEG layers produced under cloud point grafting conditions with a (−) negative control corresponding to a NeutrAvidin-coated lens deactivated with free biotin and a (+) positive control corresponding to an active NeutrAvidin-coated lens;

FIG. 5 shows an atomic force microscopy (AFM) experimental set-up: the liquid cell cross-section shows a contact lens supported on a Teflon™ mold and bulk-hydrated from the surrounding saline solution, the AFM tip accesses the anterior surface of the contact lens through a small aperture in the Teflon™ cover plate;

FIG. 6 shows 5 layers of PEG-biotinylated liposomes docked onto a soft contact lens imaged by AFM in tapping mode in saline buffer [image area 2×2 μm in 2D];

FIG. 7 shows 5 layers of PEG-biotinylated liposomes docked onto a soft contact lens imaged by AFM in tapping mode in saline buffer [image area 2×2 μm in 3D];

FIG. 8 shows aggregate layers of PEG-biotinylated liposomes docked onto a soft contact lens imaged by AFM in tapping mode in saline buffer [image area 3×3 μm in 2D];

FIG. 9 shows aggregate layers of PEG-biotinylated liposomes docked onto a soft contact lens imaged by AFM in tapping mode in saline buffer [image area 3×3 μm in 3D];

FIG. 10 shows the fraction of carboxyfluorescein (CF) remaining in liposomes immobilized on contact lens surface at ambient temperature and the kinetics of CF release from liposomes immobilized on contact lens surface at ambient temperature;

FIG. 11 shows the fraction of carboxyfluorescein (CF) remaining in 5 layers of liposomes immobilized on contact lens surface at 4, 20 and 37° C. and the kinetics of CF release from 5 layers of liposomes immobilized on contact lens surface at 4, 20 and 37° C.;

FIG. 12 shows the concentration of levofloxacin released at 37° C. by 2, 5, 10 layers of liposomes immobilized on contact lens surfaces, by contact lenses soaked overnight in a solution of levofloxacin (5 mg/ml) and by lenses bearing 10 layers of liposomes followed by an overnight soaking in a solution of levofloxacin (5 mg/ml) [errors bars correspond to standard deviations];

FIG. 13 shows the fraction of levofloxacin remaining at 37° C. in 2, 5, 10 layers of liposomes immobilized on contact lens surfaces [errors bars correspond to standard deviations];

FIG. 14 shows the fraction of levofloxacin remaining at 37° C. in contact lenses soaked overnight in a solution of levofloxacin (5 mg/ml) and in 10 layers of liposomes immobilized on a contact lens, which had been soaked in a solution of levofloxacin (5 mg/ml) [errors bars correspond to standard deviations];

FIG. 15 is a picture of a liposome-coated contact lens comprising 2 liposome layers and a control contact lens on a culture plate inoculated with Staphylococcus aureus;

FIG. 16 is a picture of a liposome-coated contact lens comprising 5 liposome layers and a control contact lens on a culture plate inoculated with Staphylococcus aureus;

FIG. 17 is a picture of a liposome-coated contact lens comprising 10 liposome layers and a control contact lens on a culture plate inoculated with Staphylococcus aureus;

FIG. 18 shows the diameter of the inhibition zone of Staphylococcus aureus versus incubation time for contact lenses with 2, 5 and 10 levofloxacin-loaded liposome layers;

FIG. 19 shows the antibacterial activity of contact lenses bearing 5 layers of liposomes loaded with levofloxacin and of control contact lenses (bearing layers of “empty” liposomes i.e., containing no levofloxacin) against Staphylococcus aureus determined by using a broth assay with an initial inoculum of 10⁴ CFU/ml [errors bars correspond to standard deviations];

FIG. 20 shows the antibacterial activity of contact lenses bearing 5 and 10 layers of liposomes loaded with levofloxacin, of contact lenses bearing 10 liposome layers soaked overnight in a 5 mg/ml levofloxacin solution, of dried contact lenses re-hydrated in a 5 mg/ml levofloxacin solution and of control contact lenses (bearing layers of “empty” liposomes i.e., containing no levofloxacin) against Staphylococcus aureus determined by using a broth assay with an initial inoculum of 10⁶ CFU/ml [errors bars correspond to standard deviations];

FIG. 21 shows the antibacterial activity of contact lenses bearing 5 and 10 layers of liposomes loaded with levofloxacin, of contact lenses bearing 10 liposome layers soaked overnight in a 5 mg/ml levofloxacin solution, of dried contact lenses re-hydrated in a 5 mg/ml levofloxacin solution and of control contact lenses (bearing layers of “empty” liposomes i.e., containing no levofloxacin) against Staphylococcus aureus determined by using a broth assay with an initial inoculum of 10⁸ CFU/ml [errors bars correspond to standard deviations];

FIG. 22 is an enlargement of the left part of FIG. 21;

FIG. 23 shows optical density values (means and standard deviations; n=3 in triplicate) for lenses bearing liposomes (lens+lipo), control lenses (ctl lens), control cultures without serum (Ctl −/s) and with serum (Ctl +s) and liposome suspensions (Ctl lipo) obtained during XTT assays on human corneal epithelial cells in direct contact with the lenses;

FIG. 24 shows the morphology of epithelial cell monolayer in the presence of serum during XTT assays on human corneal epithelial cells in direct contact with the lenses;

FIG. 25 shows the morphology of epithelial cell monolayer without serum during XTT assays on human corneal epithelial cells in direct contact with the lenses;

FIG. 26 shows the morphology of epithelial cell monolayer in contact with a lens bearing liposomes during XTT assays on human corneal epithelial cells in direct contact with the lenses;

FIG. 27 shows the morphology of epithelial cell monolayer in contact with a control lens during XTT assays on human corneal epithelial cells in direct contact with the lenses;

FIG. 28 shows corneal epithelial cell growth as detected by Hoechst staining and measured by spectrofluorometry (i.e., optical density (O.D.)) after 3 days of cultures, the cultures were in the presence of control lenses (Ctl lens), lenses bearing liposomes (Lens+lipo), in the presence of an increasing dilution of liposome suspensions (from 1/5 to 1/100), and media with serum (Ctl +/s) and without serum (Ctl −/s), n=3 in triplicate;

FIG. 29 shows elution assays with corneal epithelial cells grown in culture media that were previously soaked with the lenses after a period of 3 days, media were soaked in the presence of lenses without liposomes (black bars), lenses bearing liposomes (white bars) and investigated at different dilutions (1/1, 1/10, and 1/100) to fresh culture medium, control 3-day old media were also investigated (grey bars), the dashed lines represent the control cultures with fresh medium without serum, means and standard deviations are represented (n=3 in duplicate);

FIG. 30 shows elution assays with corneal epithelial cells grown in culture media that were previously soaked with the lenses after a period of 10 days, media were soaked in the presence of lenses without liposomes (black bars), lenses bearing liposomes (white bars) and investigated at different dilutions (1/1, 1/10, and 1/100) to fresh culture medium, control 10-day old media were also investigated (grey bars), the dashed lines represent the control cultures with fresh medium without serum, means and standard deviations are represented (n=3 in duplicate);

FIG. 31 shows in vitro reconstituted cornea/stroma, histological sections of the reconstituted cornea/sclera show the epithelial cell layer (Ep.) and the stromal layer (S.) after cultures in the presence of a control lens;

FIG. 32 shows in vitro reconstituted cornea/stroma, histological sections of the reconstituted cornea/sclera show the epithelial cell layer (Ep.) and the stromal layer (S.) after cultures in the presence or a lens bearing liposomes;

FIG. 33 shows ex vivo rabbit cornea/sclera, the rabbit cornea/sclera was in direct contact with a lens bearing liposomes as observed after toluidine blue staining, no alteration of the epithelial cell layer and the cornea was observed;

FIG. 34 shows ex vivo rabbit cornea/sclera, the rabbit cornea/sclera was in direct contact with a lens bearing liposomes as observed after carboxyfluorescein, no alteration of the epithelial cell layer and the cornea was observed;

FIG. 35 shows ex vivo rabbit cornea/sclera, the rabbit cornea/sclera was in direct contact with a lens bearing liposomes as observed by histological sections, no alteration of the epithelial cell layer and the cornea was observed; and

FIG. 36 is a comparison between the direct and indirect transmission spectra of contact lenses without liposomes and with 5 or 10 layers of stable liposomes.

### DETAILED DESCRIPTION OF ILLUSTRATIVE EMBODIMENTS

The invention relates to methods of product and surface modification, such as the surface of a contact lens, modified products/surfaces, such as contact lenses, produced by these methods, and their uses.

A “contact lens”, as used herein, refers to an optical system/device and includes hard or soft contact lenses. The contact lenses may be, for example, used as a device for localized and/or controlled drug delivery or as an in vivo analyte sensor (e.g. as a glucose sensing contact lens).

A contact lens comprises surfaces which may be defined accordingly. For example, a posterior surface may be defined as being the surface which is normally in contact with the eye when the contact lens is being worn. An anterior surface may be defined as the surface which is normally not in contact with the eye and is in contact with the air and/or the eyelid when the lens is worn.

A used herein, “low fouling” refers to preventing or hindering the deposition of a contaminating/undesirable material, such as biological material, on a surface, material or polymer. Such deposition or fouling is undesirable since it can compromise the function of the surface, material or polymer, and for example in the case of contact lenses, may affect wear comfort. For example, a low-fouling surface is one that is more resistant to the deposition of undesirable materials thereon.

In embodiments, the carrier moieties of the present invention are vesicles, such as liposomes, micelles, polymerized liposomes and polymerized micelles.

Liposome suspensions of various compositions have been described to enhance the sustained release of medications. Drug delivery by injectable liposomes and/or vesicles is known in the pharmaceutical industry. Liposomes can encapsulate in their interior some of the aqueous medium in which they are formed and which may contain an agent to be delivered.

Liposomes are microscopic vesicles having a concentric lipid bilayer structure of one or more closed concentric lamellae enclosing one or more aqueous-containing compartments. Liposomes are generally spherical and prepared from lipids or lipid-like molecules of general formula XY, wherein X is a polar hydrophilic moiety and Y is a non-polar hydrophobic moiety. The lipid or lipid-like molecules are normally arranged in a bilayered formation, similar to the lipid arrangement of biological membranes. Typically, the polar end (X) of a lipid or lipid-like molecule is in contact with the surrounding solution, usually aqueous solution, while the non-polar, hydrophobic end (Y) of the lipid or lipid-like molecule is in contact with the non-polar, hydrophobic end of another lipid or lipid-like molecule. The resulting bilayered membrane is usually selectively permeable to molecules of a certain size, hydrophobicity, shape, and/or net charge.

Liposomes can generally be divided into three categories based on their overall size and the nature of the lamellar structure. To describe these physical classifications, the nomenclature developed at the New York Academy of Sciences meeting on “Liposomes and Their Use in Biology and Medicine,” of September 1977 will be used. These three classifications are multilamellar vesicles (MLV), small unilamellar vesicles (SUV) and large unilamellar vesicles (LUV). Small unilamellar vesicles range in diameter from approximately 200 to 500 nm and consist of a single lipid bilayer surrounding an aqueous compartment. A particular characteristic of SUVs is that a large amount, about 70%, of the total lipid is located in the outer layer of the vesicle. In addition, the small radius of curvature imposes strain in packing of the lipid molecules resulting in them being rendered metastable in certain circumstances.

The most frequently encountered and easily prepared liposomes are multilamellar vesicles (MLV). MLV vary greatly in size up to about 10,000 nm and are multicompartmental in their structure.

Large unilamellar vesicles (LUV) have a diameter ranging from about 600 nm to about 30 microns. Such vesicles may contain one or more bilayers.

Liposomes may be anionic (negatively-charged surfaces), basic (positively-charged surface) or neutral depending upon the choice of hydrophilic groups. For instance when a phosphate or a sulfate group is used as the polar group (X) the resulting liposome will be anionic. When amino-containing lipids or lipid-like molecules are used, the liposomes will have a positive charge, or be cationic liposomes; and when polyethyleneoxy or glycol groups are present in the lipids or lipid-like molecules, neutral liposomes will be obtained. It should be understood that the neutral liposomes can be modified chemically or physically to have superficial charges. For example, neutral liposomes can be coated with polyelectrolytes.

Lipids or lipid-like compounds suitable for forming liposomes are commercially available and are described in for example McCutcheon's Detergents and Emulsifiers and McCutcheon's Functional Materials, Allured Pub. Company, Ridgewood, N.J., U.S.A. Exemplary lipids or lipid-like compounds include lecithin, phosphatidyl ethanolamine, lysolecithin, lysophosphatidylethanolamine, phosphatidylserine, phosphatidyl inositol, sphingomyelin, cephalin, cardiolipin, phosphatidic acid, cerebrosides, dicetyl-phosphate, phosphatidyl-choline and dipalmitoyl-phosphatidylcholine. Additional, non-phosphorous-containing lipids are for instance, stearylamine, dodecylamine, hexadecylamine, cetyl palmitate, glyceryl ricinoleate, hexadecyl stearate, isopropyl myristate, amphoteric acrylic polymers, triethanolamine-lauryl sulphate, alcoyl-aryl sulfonates, polyethoxylated fatty acid amides and the like.

Liposomes can be prepared by methods known to those of skill in the art (see, e.g., Kim et al. Bioch. Bioph. Acta 728:339-348 (1983); Assil et al. Arch Opthalmol. 105:400 (1987); Szoka & Papahadjopoulos, Ann. Rev. Biophys. Bioeng., 9:467-508 (1980); and U.S. Pat. No. 4,522,811; Liposome Technology (3^{rd} ed.), Gregory Gregoriadis, ed., CRC Press, 2006; as well other sources known to those of skill in the art). Control of the number of layers and vesicle size of liposomes is described for example in reviews by Pagano and Weinstein (Ann. Rev. Biophysic. Bioeng., 7, pp. 435-68 (1978)) and Szoka and Papahadjopoulos (Ann. Rev. Biophysic. Bioeng., 9, pp. 467-508 (1980)) as well as a number of patents (for example, U.S. Pat. Nos. 4,229,360; 4,224,179; 4,217,344; 4,241,046; 4,078,052 and 4,235,871). For example, liposome preparations may be subjected to freeze-thaw cycle(s) and/or extrusion through membranes having a defined pore size.

Various agents or materials may be encapsulated within liposomes or associated with liposomes (referred to herein as agents, guest materials or cargo). Such encapsulation for example may be achieved by preparing the liposomes in the presence of a solution of the agent. Non-encapsulated agent may be removed by subjecting the liposomes to a suitable separation or purification step, such as chromatography (e.g. gel filtration chromatography).

Various additives can be combined with the lipids or lipid-like materials so as to modify the permeability and/or superficial charges of liposomes. Representative additives include long chain alcohols and diols; sterols, for example, cholesterol; long chain amines and their quaternary ammonium derivatives; dihydroxyalkylamines; polyoxyethylenated fatty amines; esters of long chain amino alcohols, their salts and quaternary ammonium derivatives; phosphoric esters of fatty alcohols, for example, sodium dicetyl phosphate; alkysulfates, for example, sodium cetyl sulfate; certain polymers such as polypeptides; and proteins.

Liposomes may be designed and prepared to respond to a specific stimulus, or combination of stimuli, as well as to have a particular stability, rigidity, and permeability. Exemplary stimuli include, but are not limited to, pH, temperature, light, detergents, metal ions, and the like.

Furthermore, the biological stability of liposomes by modification with polymers such as polyethylene glycol (PEG) may be increased several fold as a result of the overlap of the PEG layers (i.e., steric stabilization by PEG) (Vermette P, Meager L, Colloids and Surfaces B: Biointerfaces, 28 (2003) 153-198). PEGylated liposome technology has already been approved for clinical application (e.g., the Canadian Therapeutic Products Program has approved Caelyx® [pegylated liposomal doxorubicin] for the treatment of advanced ovarian cancer in women).

Seki et al. (“Polym. Materials Sciences and Eng.”, Proc. of ACS Div. of Polym. Materials Meeting in Philadelphia, Pa., ACS, 51, 216-219 (1984)), disclose pH-dependent release of the guest materials encapsulated in liposome formed from egg yolk phosphatidyl choline. Seki et al. used a synthetic poly(carboxylic acid), poly(alphaethylacrylic acid) PEAA to effect a pH-dependent release of the encapsulated guest materials. Phosphatidyl choline vesicles are unaffected by PEAA at high pH but are rendered unstable at pH 7 or below.

Pidgeon and Hunt (“Light Sensitive Liposomes” in Photochem and Photobiol. 37, 491-494 (1983)), describe liposomes having a permeability, which can be changed by irradiation with UV light. Pidgeon and Hunt used two photosensitive phospholipids, 1,2-diretinoyl-Snglycero-3-phosphocholine and 1-palmitoyl, 2-retinoyl-Sn-glycero-3-phosphocholine, in their studies. The permeability of liposomes formed from either or both of these phospholipids is directly proportional to temperature. Upon exposure to 30 to 120 seconds of 360 nm light, the permeability of the liposomes increases dramatically, from approximately 20% to almost 90%.

Kano et al. (Photochem. Photobiol. 34, 323-325 (1981) and Chem. Lett. 421-424 (1981)), disclosed different photosensitive liposomes. Kano et al. showed that incorporation of light isomerizable azobenzene lipids into liposome membranes produces vesicles with increased membrane permeability upon exposure to light.

Sensitivity of liposomes to temperature is also well known. This is largely based on the gel-liquid crystal transition temperature (Tc or Tm) of lipids that form liposomes. A specific lipid composition may be formulated so that the transition temperature is above the temperature at which the liposomes are to encapsulate guest materials yet low enough to allow for release when the temperature is raised slightly, for example, when a contact lens modified with liposomes is being worn.

Phospholipase is able to cleave one or more of the phospholipids making up liposomes. Therefore, the liposomes formed from phospholipids may be destabilized using phospholipase enzymes so as to release guest materials encapsulated in the liposomes (i.e., liposome “cargo”).

Bivalent metals have also been shown by D. Papahadjopoulos and J. C. Watkins in Biochem. Biophys. Acta. 135, 639-652 (1967) to increase the permeability of liposomal bilayers.

A number of methods for preparing polymerized liposomes have been described (see, for example, U.S. Pat. Nos. 6,187,335; PCT International Publication WO 9503035; Chen et al., 1995, Proceed. Internat. Symp. Control. Rel. Bioact. Mater. 22; Chen et al., 1995 Proc. 3rd U.S. Japan Symposium on Drug Delivery Systems; Brey, R. N., 1997, Proc. 4th U.S. Japan Symposium on Drug Delivery;). A number of compounds have been disclosed for the formation of polymerized liposomes (see, for example, U.S. Pat. No. 4,248,829; U.S. Pat. No. 4,485,045; U.S. Pat. No. 4,808,480; U.S. Pat. No. 4,594,193; U.S. Pat. No. 5,160,740; U.S. Pat. No. 5,466,467; U.S. Pat. No. 5,366,881; Regen, in Liposomes: from Biophysics to Therapeutics (Ostro, ed., 1987), Marcel Dekker, N.Y.; Singh, A., and J. M. Schnur, 1993, “Polymerizable Phospholipids, in Phospholipids Handbook, Gregor Cevc, ed., Marcel Dekker, New York.

Polymerized liposomes, which entrap an agent, can be prepared by any method known to a person skilled in the art. For example, liposomes are first formed according to one of the above-described methods known to a person skilled in the art, to encapsulate an agent. Then, such preformed liposomes with agent encapsulated therein are polymerized by photopolymerization or thermal polymerization.

Micelles are dynamic aggregates formed in a polar solvent such as water from surfactants, molecules having both hydrophilic and hydrophobic groups. A micelle typically takes roughly the shape of a sphere, a spheroid, an ellipsoid, or a rod, with the hydrophilic groups on the exterior and the hydrophobic groups on the interior. The hydrophobic interior provides, in effect, a hydrophobic liquid phase with salvation properties differing from those of the surrounding solvent. Micelles form when the surfactant concentration in solution is greater than a characteristic value known as the critical micelle concentration (“CMC”).

Polymerized micelles, or polymerized surfactant aggregates, were first developed in the late 1970's and early 1980's. Compared to otherwise identical non-polymerized micelles (“conventional micelles”), polymerized micelles exhibit enhanced stability and better control over micelle size. An advantage of polymerized micelles is that they have no critical micelle concentration (“CMC”). A number of methods for preparing polymerized micelles have been described (see, for example, C. Palmer et al., J. High Res. Chromatogr., vol. 15, pp. 756-762 (1992); C. Larrabee et al., J. Poly. Sci.: Poly. Lett. Ed., vol. 17, pp. 749-751 (1979); D. Tabor et al., Chromatogr., vol. 20, pp. 73-80 (1989); S. Terabe et al., Anal. Chem., vol. 62, pp. 650-652 (1990); and J. Fendler et al., Acc. Chem. Res., vol. 17, pp. 3-8 (1984)).

As used herein, “agent” refers to any material, which can be associated with, entrapped/encapsulated in and/or bound to a carrier moiety. Exemplary agents include, without limitation, materials that impart desired functionalities to a medical device, for example, drugs (e.g., ophtalmic drugs, proteins (such as enzymes or hormones or the like), amino acids, nucleic acids, polypeptides, metallic nanoparticles, magnetic nanoparticles, optically active nanoparticles, dyes, biosensors, sensors (e.g., fluorescently labeled glucose receptor/adapter and the fluorescently labeled glucose competitor in ocular glucose sensors, such as that disclosed in PCT International Publication WO 01/13783).

A “sensor” is an agent capable of providing a detectable signal in the presence (or absence, depending on its mode of function) of a molecule or parameter to be assessed. Such a signal may for example be based on the binding/interaction of the sensor to the molecule and/or a change in the properties/activity of the sensor in the presence of the molecule or parameter to be assessed.

As used herein, the term “biosensors” refers to any sensor device or system that is partially or entirely composed of biological molecules (such as enzymes, antibodies, whole cells, organelles, or combinations thereof).

As used herein the term “drugs” includes medicaments, therapeutics, vitamins, nutritional supplements, and the like. If the guest material is a drug, it may be present in therapeutically effective amounts relative to its function as can be determined by the person of skill in the art.

Any pharmaceutical drug can be utilized such as, for example, anti-cancer drugs, drugs for allergy, hormone drugs, antibiotics, drugs for chemotherapy, vitamins, food supplements and the like.

Agents, such as drugs, can be delivered using a carrier moiety or modified contact lens device once it is in contacted with a tissue or body fluid. If the drug is covalently linked to the carrier moiety, it may for example be released by enzymatic cleavage (hydrolysis). Alternatively, the encapsulated or associated drug may be released from the carrier moiety after contact with the tissue or body fluid. Further, in embodiments the agent may impart its activity in a localized manner while associated with the carrier moiety.

In embodiments, the rate of release of the agent may vary. For example, the agent may be released rapidly, or in a more slow, controlled release fashion. Further, multiple rates of release may be utilized, such as an initial rapid release followed by a slower prolonged rate of release. In embodiments, the agent may be released in less than 1 hr, in further embodiments, over a period of time of at least 1, 2, 3, 4, 5, 10, 20, 30, 40, 50, 60, 80, 100, 120, 140 or 280 hours.

The carrier moieties, e.g. vesicles, are attached to the surfaces of a contact lens using specific ligand-receptor (an adapter molecule) interactions.

The attachment of the vesicles or carrier moieties does not proceed by way of ionic interactions as those involved in layer-by-layer processes using polyelectrolytes. Rather, the first and the second ligands described herein bind to the receptor or adapter to form a complex. Ligands are molecules that bind to a binding site on a receptor (or adapter) surface by intermolecular forces (non-covalent). The first and second receptor or adapter of the invention are multivalent, i.e. have two or more binding sites capable of concurrently binding both the first and second ligands and/or two second ligands so that the carrier moiety will become attached to the surface of the contact lens and/or two carriers moieties will become attached to each other. The first and second ligands may be the same or may be different; in the latter case the adapter has the ability to bind the different ligands. As such, the adapter may have homospecific or heterospecific binding activity. The first and second adapter molecules may be the same of different.

Any ligand-receptor/adapter system known to interact with each other can be used herein. It is preferred that the ligands used have relatively high binding affinity with the receptor/adapter so as to have a long residence time at their receptor/adapter binding sites and form a stable complex. An example of a ligand-adapter system is that of the ligand biotin (or a related ligand) and a corresponding biotin-binding protein (e.g. a protein of the avidin family) as an adapter molecule.

Non-limiting examples of ligands and receptor/adapters are shown below:

| Ligands | Receptors/adapters |
|---|---|
| biotin, iminobiotin | avidin, streptavidin, NeutrAvidin ™ |
| carbohydrates | Concavalin A or lectin |

Metal-ion complexes may also be used as ligand-adapter system.

In the present invention, there may be one or more “layers” of carrier moieties bound to the contact lens. The term “layers” is used loosely here as these “layers” need not result in a regular layer structure. The first and second carrier moieties may be the same or may be different in nature or in content. It is contemplated that both these carrier moieties may be a mixture of carrier moieties that are different in nature or in content.

In the context of the present invention, the first ligands may be covalently linked to the contact lens either directly or by the way of one or more intermediate molecules, such as different polymers, which are covalently linked together and are covalently linked to the surface of lens and to the first ligand.

The second ligands may be attached to the carrier moieties by any means known to the person of ordinary skill in the art.

In the context of the present invention, the expression “incubating the contact lens in the presence of”, for example, a coupling agent means to put the contact lens in contact with the agent, for example by contacting the contact lens with a liquid media containing the coupling agent, for a period of time sufficient for the agent to interact with the contact lens and modify it in some way. In embodiments, it is possible to modify only some of the surface(s) of a contact lens, for example by protecting the other surface(s) from exposure to the incubating medium and the modifying reagent.

The carrier moieties may be, in some embodiments, in the form of aggregates. Aggregates of the carrier moieties may for example comprise clusters or two or more carrier moieties linked together by two second ligands and an adapter molecule.

The first and second functional groups may be any functional group. In embodiments, which react to form a covalent link. More specifically, these first and second functional groups may be amine or ester groups. The second functional groups are either part of the backbone of the polymer or of its side chains.

The polymer may be any polymer which comprises functional groups and allows the modified surface or contact lens to be safely used for its intended purpose. Such polymer may be a polyethylene glycol (PEG)-based polymer, for example, NHS-PEG-biotin. Also, such polymers may be low-fouling. More specifically, the polymer may be carboxymethyl dextran.

In the context of the present invention, “cloud point” conditions, which may also be referred to as theta solvent conditions or salting out, refer to conditions under which a polymer agglomerates or aggregates based on the principle that some polymers undergo agglomeration or aggregation at certain (e.g. high) salt concentrations and/or at certain temperature conditions. The type of salt, the salt concentration, and/or the temperature conditions needed for the polymer to agglomerates/aggregates differs from polymer to polymer. Such a solution can be obtained by first dissolving the polymer in an adequate solvent and then adding one or more salts to the solution and/or changing its temperature until a cloudy appearance, which is indicative of the presence of polymer aggregates, is obtained.

The third functional groups may be any functional groups that can be activated by a coupling agent. More specifically, the functional groups may be the functional groups normally present on the surface of commercially sold contact lenses. The functional groups may be, for example, hydroxyl or carboxyl groups.

In the context of the present invention, “activating” a functional group means to modify the functional groups to facilitate its reaction with a further reactant.

The coupling agents are agents that react with for example the third functional groups present on the contact lens to form a new functional group that will be more readily able to react with a further reactant. Non limiting examples of coupling agents include those based on carbodiimide or shift base chemistry. In an embodiment, the coupling agent may be may a hydroxyl-reactive coupling agent. Such agent may be N,N′-disuccinimidyl carbonate (DSC), epoxides, oxiranes, oxidation with periodate, enzymatic oxidation, alkyl halogens, isocyanates, or carbonyldiimidazole (CDI), which are also able to react with and activate other types of functional groups.

Various ligands, adaptors, coupling agents, etc. may be combined and incubated under conditions facilitating the desired interaction, reaction, etc., e.g., suitable temperature, pH, time, mixing/agitation, and/or presence of suitable salt/ionic strength/buffer/other factors. Suitable e.g., wash, separation, purification, sterilization, etc. steps may also be used, as appropriate, in the methods of the invention.

The present invention is illustrated in further details by the following non-limiting examples.

### Example 1

### Preparation of Surface Modified Contact Lenses

Soft contact lenses have been developed as vehicles for ophthalmic drug delivery and/or as biosensors. Drugs or sensing agents are encapsulated in liposomes and then subsequently these intact lipid vesicles are bound onto contact lenses.

First, polyethylenimine was covalently attached onto the hydroxyl groups available on the surface of a commercial contact lens (Hioxifilcon B). Then, NHS-PEG-biotin molecules were covalently attached onto surface amine groups by carbodiimide chemistry.

Intact liposomes were immobilized onto the surface of contact lenses using both a monolayer and a multilayer immobilization strategy. To do so, NeutrAvidin protein molecules were bound onto the PEG-biotin layer. Finally, liposomes containing PEG-biotinylated lipids were docked onto the remaining sites of the surface-immobilized NeutrAvidin molecules.

Multilayers of liposomes were produced by two methods: (1) consecutive addition of further NeutrAvidin and liposome layers and (2) exposing the contact lenses coated with NeutrAvidin to liposome aggregates produced by the addition of free biotin in the liposome solution.

Materials Used for the Preparation of the Surface Modified Contact Lenses

Contact lenses (Hioxifilcon B, Opti-Gel 45G, Opti-Centre, Sherbrooke, QC, Canada) were used as substrates for surface immobilization of intact liposomes.

Hexane (A.C.S. grade) was purchased from ACP (Montreal, QC, Canada).

Disuccimidylcarbonate (DSC)— the activation can also, if desired, be made by using either epoxides, oxiranes, oxidation with periodate, enzymatic oxidation, alkyl halogens, isocyanates, or carbonyldiimidazole (CDI)— anhydrous acetonitrile (CH₃CN, 99.9% purity, #271004), N-hydroxysuccinimide (NHS, #H-7377), N-[2-hydroxyethyl]piperazine-N′-[2-ethanesulfonic acid] (HEPES, #H-3375) (99.5%), t-octylphenoxypolyethoxyethanol (Triton X-100, #T-9284), 1-ethyl-3-(3-dimethylaminopropyl)carbodiimide (EDC, #E-1769), tris(hydroxy methyl amino methane) (TBS, Trizma, # T1503) and 4-nitrophenyl phosphate disodium salt hexahydrate (#9389) were purchased from Sigma-Aldrich (Oakville, ON, Canada).

Poly(ethylenimine) (PEI, 70 kDa MW, #00618) was obtained from Polysciences Inc. (Warrington, Pa., USA).

Sodium chloride (NaCl) (A.C.S. grade), sodium sulfate (Na₂SO₄) (A.C.S. grade) and chloroform (A.C.S. grade) were purchased from Fisher Scientific (Ottawa, Ontario, Canada).

1,2-distearoyl-sn-glycero-3-phosphocholine (DSPC) (>99%, #850365), cholesterol (CHOL) (>99%, #700000), N-[ω-(biotinoylamino)poly(ethylene glycol) 2000]-1,2-distearoyl-sn-glycero-3-phosphoethanolamine (DSPE-PEG(2000)-Biotin) (>96%, #890129) were obtained from Avanti Polar Lipids Inc. (Alabaster, Ala., USA).

Biotin-PEG-NHS(NHS-PEG-Biotin, #0H4M0F02) was purchased from Nektar Therapeutics (Huntsville, Ala., USA).

5-(and-6)-carboxyfluorescein (CF, mixed isomers, 99%, C194) was obtained from Molecular Probes (Eugene, Oreg., USA).

Biotinylated calf intestinal alkaline phosphatase (biotin-AP, ImmunoPure biotinylated alkaline phosphatase, #0029339) was purchased from Pierce (Rockford, Ill., USA).

NeutrAvidin (ImmunoPure® NeutrAvidin™ biotin-binding protein, #P31000) and D-biotin (ImmunoPure® D-Biotin, #29129) were obtained from Pierce (Rockford, Ill., USA). NeutrAvidin is a modified avidin with low non-specific binding properties and does not contain carbohydrate, thus eliminating the potential of binding to lectins.

Preparation of Liposomes

The buffer used to prepare the liposome suspensions contained 10 mM HEPES at pH 7.4 and a NaCl concentration adjusted to an osmolarity of 290 mOsm using the Advanced™ osmometer, model 3250 (Advanced Instruments Inc., Norwood, Mass., USA). Milli-Q gradient water (Millipore Canada, Nepean, Ontario, Canada) with a resistivity of not less than 18.2 MΩ·cm was used to prepare the buffer solutions. The buffer solution was filtered using sterile 0.22-μm filters (Millex® GP, Millipore, Cork, Ireland) before use in the preparation of the liposome suspensions.

Unilamellar vesicles (ULVs) were prepared by first mixing in a round-bottom flask DSPC, cholesterol, and DSPC-PEG(2000)-Biotin (2:1:5 mol % mol ratios) in HPLC-grade chloroform. Approximately 144 μmol of lipids were deposited from 2 ml HPLC-grade chloroform solution to form a thin film on the interior surface of a 50 mL round-bottom flask by rotary evaporation under a pressure of ˜13 332 Pa for 4 h. Following addition of HEPES buffer, lipids were hydrated in the dark above 65° C. The multilamellar vesicle (MLV) suspension thus produced was subjected to ten freeze-thaw cycles involving quenching in dry ice and in acetone, followed by immersion in a 65 LC water-bath. Unilamellar vesicles were finally produced by extrusion through 100-nm pore polycarbonate Avestin® track-etch membranes using the Avestin Liposofast (Avestin Inc., Ottawa, Ontario, Canada) operated at 65° C.

Immobilization of the Liposomes

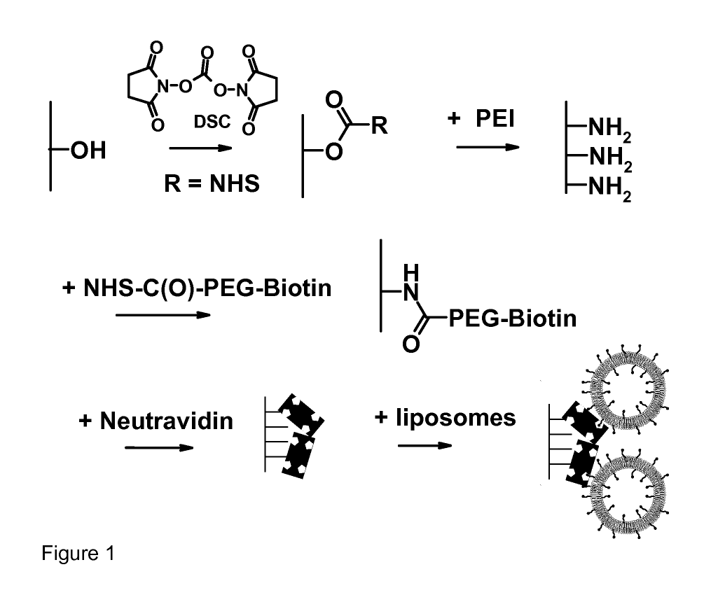

The contact lens surface modification strategy is described in FIG. 1.

To remove any chemical contaminants and to condition the contact lens structure, each contact lens was first sonicated in 4 mL of hexane for 15 min three times. After this pre-washing step, the remaining hexane was evaporated under vacuum for 2 hours. Each contact lens was then sonicated in 4 mL of anhydrous acetonitrile for 15 min to remove any trace of contaminants. Anhydrous acetonitrile was used to avoid the swelling of the contact lens material prior to the first chemical surface modification. It was verified by Scanning Electron Microscopy (SEM) that the contact lens structure was not affected by this washing step in acetonitrile (data not shown here). The contact lens material is composed of two polymers, the poly-hydroxyethyl methacrylate (PHEMA) and the poly-glycidyl methacrylate (pGMA).

The surface modification strategy consisted of the activation of the hydroxyl groups of the pHEMA by a coupling reagent, N,N′-disuccinimidyl carbonate, in excess in anhydrous acetonitrile. Activation of hydroxyl groups can also, if desired, be made by using either N,N′-disuccinimidyl carbonate (DSC), epoxides, oxiranes, oxidation with periodate, enzymatic oxidation, alkyl halogens, isocyanates, or carbonyldiimidazole (CDI). Acetonitrile was used as a solvent because it did not cause swelling of the contact lens and limited the possibility of cross-linking. Each contact lens was immersed in 3 ml of this DSC solution for 1 hour under vigorous shaking.

Then, to provide a surface with a good density of amino groups on the contact lens surface, the lenses were rinsed in acetonitrile and immersed in a 3 mg/ml solution of polyethylenimine (PEI) in Milli-Q water with the pH adjusted to 7.4 with 1 M HCl. The reaction was allowed to proceed overnight under vigorous shaking. To remove any non-covalently adsorbed PEI, the contact lenses were then rinsed overnight under vigorous shaking in a 150 mM NaCl solution with the solution changed twice. The lenses were finally soaked overnight in Milli-Q water with the solution changed twice prior to further use.

NHS-PEG-Biotin was grafted onto the PEI layer using water-soluble carbodiimide chemistry using two approaches. In the first approach, PEI-coated lenses were immersed in a 1 mg/ml solution of NHS-PEG-Biotin. Since the active ester group on the NHS-PEG-Biotin molecule undergoes hydrolysis, 0.7 mg ml⁻¹ of EDC and NHS was added to the NHS-PEG-Biotin solution during the coupling procedure to re-activate the ester groups that could be potentially hydrolysed.

The second approach involved that the NHS-PEG-Biotin was attached onto PEI-coated lenses using cloud point conditions. Under these immobilization conditions, 170 mg·ml⁻¹ of Na₂SO₄ were added to the solution of NHS-PEG-Biotin in order to form aggregates of PEG in the solution and thus, to increase the density of PEG on the surface of the contact lenses. These conditions of cloud point were selected because by using Na₂SO₄ at this concentration there is no need to heat the solution. The reaction was allowed to proceed overnight at room temperature under vigorous shaking. To remove any non-covalently attached NHS-PEG-Biotin, the contact lenses were then rinsed overnight under vigorous shaking in a 150 mM NaCl solution with the solution changed twice. The lenses were finally soaked overnight in Milli-Q water with the solution changed twice.

Next, PEG-Biotin-coated contact lenses were immersed in a 50 μg/ml solution of NeutrAvidin in 10 mM HEPES buffer at pH 7.4. The reaction was allowed to proceed overnight at room temperature. To remove any proteins not linked to PEG-biotin (i.e., NeutrAvidin molecules that can be loosely adsorbed onto the PEG layer), samples were rinsed overnight in a 10 mM HEPES solution with the solution changed twice.

Immobilization of liposomes was performed by incubating the NeutrAvidin-coated lenses in a 1 mg/ml (total lipid concentration) biotinylated-liposome suspension made of DSPC:CHOL:DSPE-PEG(2000)-Biotin (2:1:5 mol %) for 1 h. NeutrAvidin-coated contact lenses were immersed in 3 ml of the biotinylated-liposome suspension. To remove loosely adsorbed liposomes, the samples were rinsed for 1 hour in the HEPES buffer. The buffer solution was changed three times. Multi-layers of liposomes were fabricated using two methods:

Firstly, by adding, after the attachment of the first liposome layer, more NeutrAvidin, which can add to biotins on the solution side of the liposomes present, following which more liposomes can be added to bind onto the NeutrAvidin molecules, and so forth. Contact lenses bearing one layer of surface-immobilized biotinylated liposomes were immersed in a 50 μg/ml solution of NeutrAvidin in 10 mM HEPES buffer at pH 7.4 for 30 minutes. They were rinsed for 1 hour in the HEPES buffer with the solution changed three times. A next layer of liposomes was then added to the surfaces by incubating them in the 1 mg/ml biotinylated-liposome suspension.

Secondly, by exposing NeutrAvidin-coated lenses to biotinylated liposome aggregates. Liposome aggregation via NeutrAvidin-biotin interactions was induced by adding an aliquot of a NeutrAvidin solution to a liposome suspension at a fixed ratio of receptor/adapter (NeutrAvidin) to DSPE-PEG(2000)-biotin. The ratio leading to the largest aggregates with a liposome suspension of 1 mg·mL⁻¹ was previously determined to be 0.4. Therefore, we decided to use this ratio. Contact lenses were exposed to 900 μL of a 1 mg·mL⁻¹ suspension of liposomes for 10 min followed by the addition of 900 μL of a 2 mg·mL⁻¹ NeutrAvidin solution. Samples were gently shaken after addition of the protein solution to ensure thorough mixing. The suspensions appeared cloudy within a few seconds following the addition. The reaction was then allowed to proceed for 24 h in the dark.

The term “layers” is used loosely here as we do not envisage a regular layer structure when adding NeutrAvidin solution followed by additional biotinylated liposomes to the first liposome layer. With the use of a multi-layer strategy, the loading capacity for drugs can be extended substantially beyond that achievable with one surface-immobilized layer of liposomes should this be necessary by repeated application of layers of NeutrAvidin and biotinylated liposomes. The liposome multi-layer concept may also be useful for the simultaneous delivery of several therapeutic, or, on account of the ensuing diffusional limitations, slower release of larger drugs from lower liposome layers.

Elemental Composition of the Surface-Modified Contact Lenses by X-Ray Photoelectron Spectroscopy (XPS)

XPS was performed using an AXIS HS spectrometer (Kratos, UK) equipped with a monochromatic Al Kα source at a power of 120 W. The pressure in the main vacuum chamber during analysis was typically 5×10⁻⁸ mbar. Elements present were identified from survey spectra. High-resolution spectra were collected at 40 eV pass energy (yielding a typical peak width for polymers of ca. 1 eV). Atomic concentrations of each element were calculated by determining the relevant integral peak areas, and applying the sensitivity factors supplied by the instrument manufacturer. A Shirley background was used. A reference binding energy of 285.0 eV for the ‘neutral’ C signal was used to correct for offsets due to incomplete charge neutralisation of specimens under irradiation (typically ≈3 eV in this case).

To remove salts from the contact lenses that have been exposed to HEPES buffer, the lenses were briefly rinsed in Milli-Q water prior to XPS analyses and blown dry using a high-velocity stream of 0.2-μm filtered air.

The steps in the progressive construction of the multilayer coatings were assessed by XPS analyses. The elemental compositions determined by XPS are listed in Table 1.

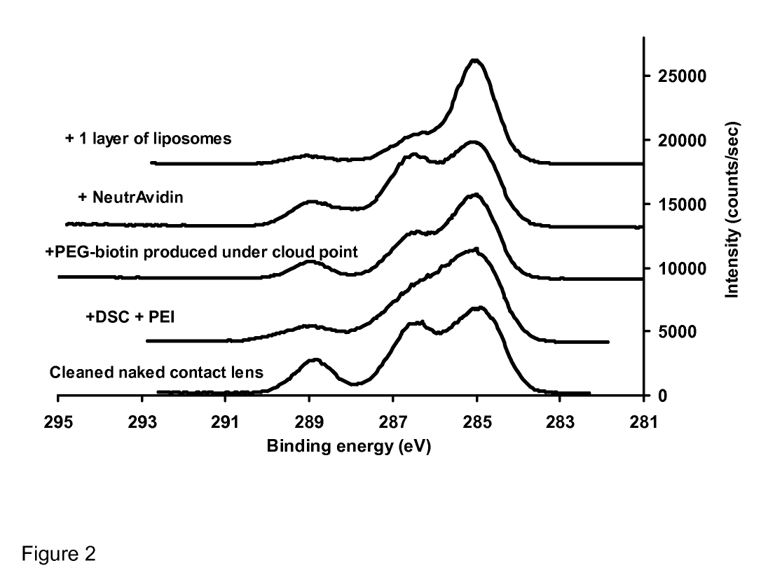

The elemental composition of the contact lens surface is in agreement with the elemental composition of hioxifilcon polymer. The lens surface is dominated by oxygen and carbon species. There were significant peak shifts in the C is spectrum (FIG. 2) due to the presence of a variety of oxygen-carbon species within the polymer structure with the most clearly defined shifts caused by C—O at 286.5 eV and C—O—C═O at 289 eV; such multi-functionality is typical of the two polymers composing the hioxifilcon material.

The grafting of PEI onto the contact lens surfaces resulted in a substantial increase of the N signal (Table 1). The XPS C 1s spectrum recorded after PEI grafting is also shown in FIG. 2 and shows an increase in the spectral region where C—N species are expected (286.5 eV). This XPS C 1s spectrum does not indicate the presence of carbonate species in the region of 290.5 eV, this demonstrates that the activation of the hydroxyl groups by DSC does not lead to cross-linking. DSC, the coupling agent, was employed in excess and the swelling of the contact lens was avoided by using anhydrous acetonitrile: the solvent and coupling agent penetration was limited, thus, the activation was mainly obtained on the contact lens surfaces. The elemental concentrations indicate that the PEI coating is extremely thin.

FIG. 2 and Table 1 show the effect on the elemental composition (XPS) of the attachment of PEG-Biotin onto the PEI layer. The presence of PEG-biotin is indicated by the increase in the C—O contribution at 286.5 eV (FIG. 2). The theoretical oxygen content for a pure PEG coating of >10 nm thickness is 33%; thus, the XPS data revealed much thinner PEG layers. The results obtained with and without cloud point grafting are compared both in Table 1 and Table 2. From Tables 1 and 2, it is shown that both the oxygen content and the C—O contribution increased for PEG layers produced under cloud point conditions as compared to PEG layers produced without cloud point conditions. With the use of cloud point conditions, the oxygen content of the PEG coating reached ca. 23%.

TABLE 1
Elemental composition determined by XPS of samples at various
stages of the multi-stage coating process on contact lenses.

| Sample | % C | % O | % N | % P |
|---|---|---|---|---|
| Lens after cleaning with hexane | 69.75 | 29.20 | 0.00 | 0.00 |
| Lens + DSC-PEI | 72.78 | 20.89 | 3.43 | 0.00 |
| Lens + DSC-PEI + PEG-biotin | 73.73 | 19.98 | 4.22 | 0.00 |
| Lens + DSC-PEI + PEG-biotin | 72.12 | 23.34 | 3.36 | 0.00 |
| (cloud point) | | | | |
| Lens + DSC-PEI + PEG-biotin + | 72.64 | 22.43 | 3.59 | 0.00 |
| Neut | | | | |
| Lens + DSC-PEI + PEG-biotin | 67.26 | 26.06 | 5.91 | 0.00 |
| (cloud point) + Neut | | | | |
| Lens + DSC-PEI + PEG-biotin | 80.91 | 15.15 | 3.09 | 0.84 |
| (cloud point) + Neut + 1 layer | | | | |
| of liposomes | | | | |

Following the addition of NeutrAvidin and washing, a substantial amount of protein was detected. High-resolution XPS C 1s spectra showed an increase in the component at the spectral position indicative of amide groups (288.5 eV); FIG. 2 contains a representative example. The increase in the N content detected on NeutrAvidin-coated surfaces over those of the PEG-Biotin-coated samples (Table 1) also indicated the presence of immobilized proteins. At this step, the influence of cloud point grafting was reinforced: the nitrogen content of NeutrAvidin immobilized on PEG layers produced under cloud point conditions was greater than that of NeutrAvidin immobilized on PEG layers produced without cloud point conditions (Table 1). The C—O contribution was also enhanced (Table 2) for NeutrAvidin immobilized on PEG layers produced under cloud point conditions compared to NeutrAvidin attached to PEG layers produced without cloud point conditions.

TABLE 2
C—C and C—O XPS intensities ratio of samples produced
with PEG layers with and without cloud point.

| Samples | (C—O)/(C—C) |
|---|---|
| Lens + DSC-PEI + PEG-biotin | 0.48 |
| Lens + DSC-PEI + PEG-biotin (cloud point) | 0.75 |
| Lens + DSC-PEI + PEG-biotin + Neut | 0.54 |
| Lens + DSC-PEI + PEG-biotin (cloud point) + Neut | 0.88 |

After ‘docking’ biotinylated liposomes onto NeutrAvidin-coated samples and washing to remove loosely adsorbed vesicles, a substantial amount of lipids were detected by XPS. In fact, the spectrum is dominated by contributions assignable to the lipids used. The presence of a phosphorus signal (Table 1) also confirms the presence of phospholipids, which is in agreement with results obtained on FEP surfaces.

Further liposome layers can be built up by adding, after the attachment of the first liposome layer, more NeutrAvidin, which can bind to free biotins on the first liposome layer, followed by addition of more liposome suspension. Thus, NeutrAvidin molecules act as ‘bridges’ between liposome ‘layers’.

Detection of NeutrAvidin Activity by Enzyme-Linked Immunosorbent Assay (ELISA)

By blocking with excess biotin surface-immobilized NeutrAvidin on the contact lenses bearing PEG-biotin layers produced under cloud point conditions, ELISA assays showed that the docking of NeutrAvidin was dependent largely on biotin-NeutrAvidin affinity binding, with little evidence for non-specific physisorption.

ELISA assays were used to probe whether NeutrAvidin molecules might have been surface immobilized by effects other than the intended affinity binding on the PEG-biotin coating. Comparison was made by “de-activating” NeutrAvidin with excess D-biotin (10 mM D-biotin made in 10 mM HEPES buffer, pH 7.4) prior to applying it to the PEG-biotin coating.

Biotinylated calf intestinal alkaline phosphatase (biotin-AP) was used to detect NeutrAvidin. Biotin-AP was diluted to 10 units/ml in 10 mM TBS and 150 mM NaCl at pH 7.5 and stored in 0.5-ml Eppendorf tubes at −80° C. until needed. The contact lenses were rinsed 10 times with TBS. The biotin-AP was added (0.5 ml/well) at a concentration of 1/100 unit/ml (made in TBS buffer, pH 7.5) on the contact lenses coated with NeutrAvidin and on the negative controls “de-activated” with excess D-biotin and allowed to react for 1 h. The lenses were rinsed four times with TBS buffer. The substrate, 4-nitrophenyl phosphate disodium salt hexahydrate, was then added (0.5 ml/well) at a concentration of 1 mg/ml (made in TBS buffer, pH 7.5) and incubated for 20 min.

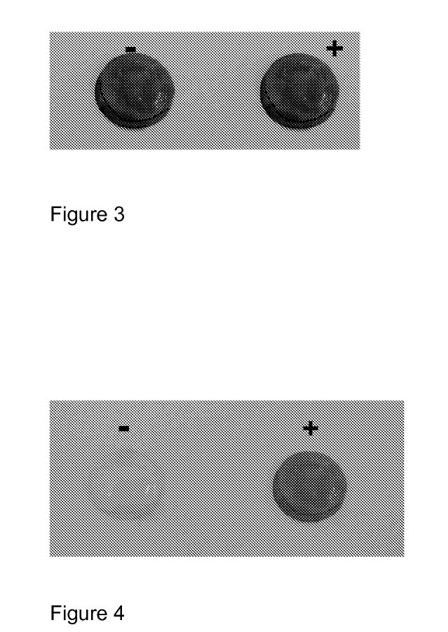

The results obtained from the ELISA assays, shown in FIGS. 3 and 4, demonstrate that biotin-AP enzyme activity was observed on three samples and that the activity of immobilized NeutrAvidin toward biotin-AP was affected by the method of fabrication of the PEG layers. The total activity of the NeutrAvidin molecules immobilized on PEG layers produced without cloud point conditions was greater than the activity of the NeutrAvidin molecules immobilized on PEG layers produced under cloud point conditions.

De-activated NeutrAvidin-coated lenses produced without the use of the cloud point conditions showed similar protein activities to the samples non-deactivated with free biotin: this observation suggests that there was non-specific adsorption and/or absorption of the protein molecules (either the NeutrAvidin and/or the AP-biotin) onto and/or within the lenses structure.

By cutting the lenses, colour change was observed all through the lens thickness for the contact lenses coated with PEG layers produced without cloud point conditions while colour change was only located on both surfaces of the lenses for contact lenses bearing PEG coatings produced under cloud point conditions. This indicated that the PEG layers produced under cloud point conditions are able to limit non-specific adsorption of the NeutrAvidin molecules onto the contact lenses surfaces and/or to hinder the penetration of the NeutrAvidin molecules within the lenses structure. In either way, the ELISA assays revealed that the detected NeutrAvidin molecules onto the surfaces of contact lenses bearing PEG layers grafted using cloud point conditions were specifically bound to the contact lenses coated with PEG-biotin layers.

With regard to the XPS analyses and ELISA assays, lenses coated with PEG layers produced under the cloud point grafting conditions were used for the following experiments involving liposome immobilization.

Imaging of Surface-Immobilized Layers of Intact Liposomes by Atomic Force Microscopy (AFM)

AFM was successfully used to image layers of intact liposomes immobilized on contact lens surfaces. AFM revealed liposome sizes of approximately 120 and 180 nm for layers of liposomes produced (i) by the consecutive addition of further NeutrAvidin and liposomes and (ii) by the exposure of NeutrAvidin-coated contact lenses to liposome aggregates, respectively.

Atomic Force Microscopy was performed using a Digital Nanoscope IIIa Bioscope (Veeco Instruments, Santa Barbara, Calif., USA). All imaging was performed via tapping mode with oxide sharpened silicon nitride cantilevers with integrated pyramidal tips (Model DNPS, Veeco NanoProbe Tips). Tapping mode imaging greatly reduces the magnitude of lateral forces applied to samples and appears more appropriate for imaging liposomes. Another advantage is that tapping mode is less sensitive to drift of the cantilever. The drive frequencies were chosen between 7.8 and 8.1 kHz. The RMS amplitude was fixed at 0.3 V. Cantilevers used in this study have a spring constant of 0.32 N/m.

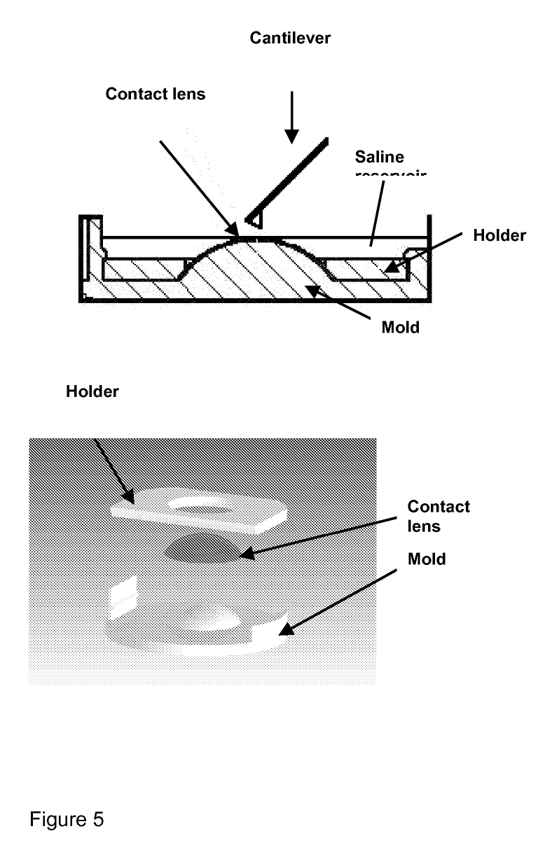

To analyze a contact lens in a liquid environment, a liquid cell adapted to receive an entire contact lens was designed and used (FIG. 5). In this cell, a contact lens is supported on a contoured Teflon™ mold. Surrounding the mold is a liquid reservoir filled with a NaCl solution (150 mM), which is used to fully immerse the contact lens in liquid. A cover plate, with a 12-mm aperture, was machined to hold firmly the contact lens on the Teflon™ mold without distorting the material. The AFM tip accesses the immersed contact lens through the aperture.

The contact lens and the cantilever were allowed to equilibrate for 1 h prior to AFM experiments. Imaging was performed in a glass fluid cell with ports so that the fluid in the chamber could be easily exchanged with the sample and the cantilever already mounted. Repeatability of the images was ascertained; representative images are presented.

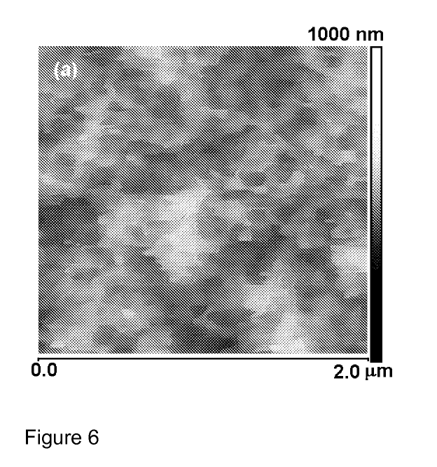

Tapping mode is known to reduce frictional and adhesive forces that often interfere with imaging of soft samples and is therefore used in this study. Typical amplitude images recorded following the immobilization of 5 biotinylated liposomes layers or liposome aggregates onto contact lens coated with PEI+PEG-Biotin+NeutrAvidin layers are shown in FIGS. 6 to 9.

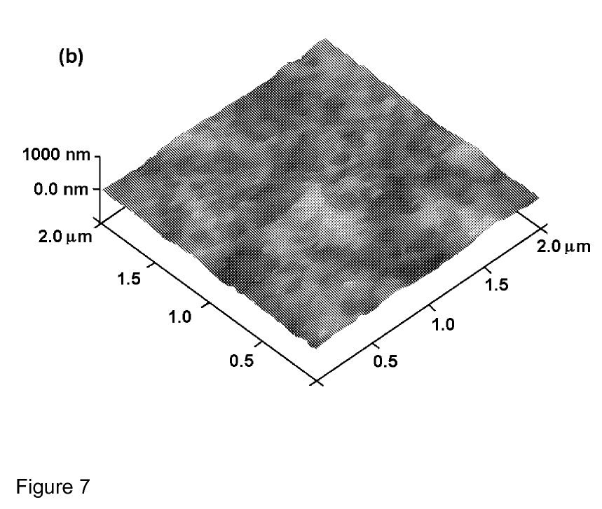

FIGS. 6 and 7 show several individual liposomes (spherical particles) in 2D and 3D views, respectively. The average diameters of these particles was measured by AFM images sectional analysis and was found to be 106±7 nm (n=20). This result is in good agreement with the average diameter measured by dynamic light scattering of liposomes extruded with a pore membrane of 100 nm.

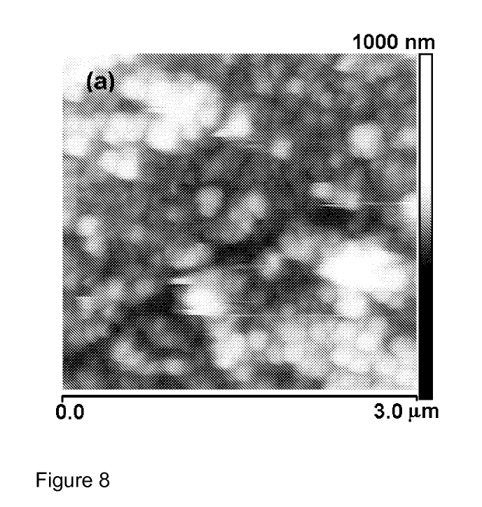

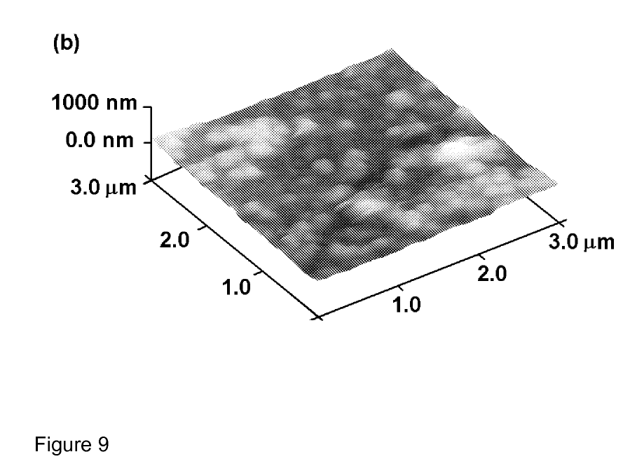

FIGS. 8 and 9 resolve several liposome aggregates (large spherical particles) on a first layer of liposomes (small spherical particles) in 2D and 3D views, respectively. The average diameter of the aggregates was estimated to be 155±7 nm (n=20) whereas the diameter of the liposomes underneath was approximately 106 nm as in the multilayer case. This value is half the average diameter reported for liposomes aggregates at the same NeutrAvidin/DPSE-PEG-Biotin ratio in solution. This difference can be explained by the formation of the aggregates directly on the contact lens surface that is composed, prior to the exposure of the liposome+NeutrAvidin solution, of a final layer of NeutrAvidin. The liposomes seem to form a first layer of individual lipid vesicles on the contact lens surface before the aggregation process occurs. Thus, the diameter of the aggregates could be decreased by sterical effects owing to the contact lens surface.

Stability of Surface-Immobilized Layers of Intact Liposomes by Fluorescence Release Measurement

The release kinetics of a fluorescent dye also demonstrated that intact liposomes had been successfully immobilized onto contact lenses surfaces. The stability of surface-immobilized liposomes onto contact lenses surfaces showed temperature dependence. Our study revealed that liposomes can be stored up to one month at 4° C. with little release of their content.

For the stability experiments, liposomes were produced with the lipids hydrated in the dark using a solution containing 85 mM CF and 10 mM HEPES at pH 7.4. The osmolarity of this CF solution was determined to be approximately 290 mOsm. Unilamellar vesicles were then produced as described in Section 3.2.1.

Separation of the dye-containing vesicles from non-entrapped CF was achieved by gel chromatography, which involved passage through a 2.5×25 cm column of Sephadex™ G-50 Fine (Amersham Pharmacia Biotech, Québec, QC, Canada). The column was eluted at room temperature with HEPES buffer. The total lipid concentration of the liposome suspension collected at the column outlet was adjusted to the desired concentration using HEPES buffer.

Liposomes containing CF were immobilized onto NeutrAvidin-coated contact lenses and the release of CF from liposomes was monitored over time using a Bio-Tek Synergy HT well-plates reader (Bio-Tek. Instruments, Winooski, VE, USA). Then, 3 ml of the appropriate medium (either 0.5% w/v Triton X-100 solution made with Milli-Q water or HEPES buffer) was added to each vial containing a contact lens bearing layers of immobilized liposome and incubated at 4° C., 20° C. or 37° C. At low concentration and neutral pH, Triton X-100 instantaneously disrupts the vesicles and liberates their contents without significant interference with the intrinsic fluorescence of the CF dye (significant interference can occur at lower pH or in the presence of serum components).

At periodic intervals, 200 μL of the incubating solution was withdrawn from the vial containing the lenses and transferred into 96-well plates for fluorescence measurement. The samples of 200 μL were then returned to the vial containing the lens to maintain a constant volume for the whole experiment. The total fluorescence is determined after disrupting the remaining vesicles with the detergent Triton X-100. CF has an excitation maximum at 487 nm at pH 7.5 and an emission maximum at 520 nm. The fluorescence signal was monitored at 520 nm. Each experiment was done in triplicate. The CF release over time was calculated using Equation 1:
Fraction of CF remaining in vesicles=1−F/FT (Eq. 1)
where F is the fluorescence at 520 nm measured at any time during the experiment, and F_{T} is the total CF fluorescence at 520 nm determined after disruption of the vesicles with Triton X-100.

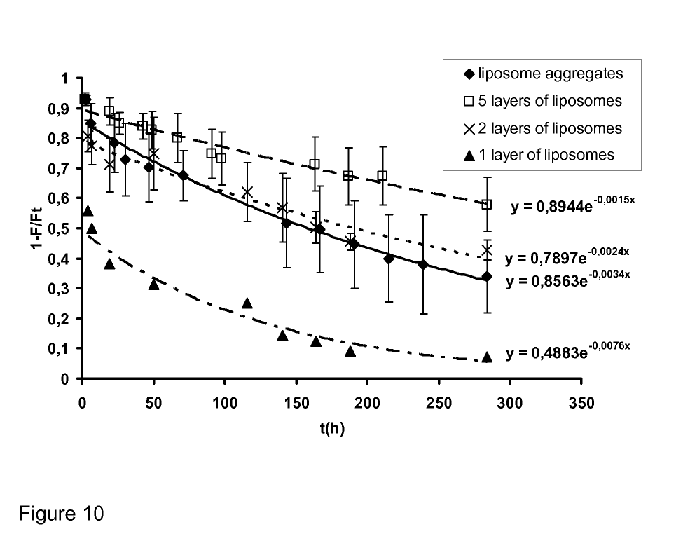

The release kinetics of CF is shown in FIG. 10. On the molecular scale, the course of the release of CF may be complex, but the form of the empirical rate law shown in FIG. 10 suggests that the particular path via which the release of CF takes place follows a first order release model. This suggests that CF was released by diffusion rather than disruption of the liposomes.

To compare the release kinetics of each lens, the collected data were analyzed by an exponential function. At ambient temperature, it was found that liposomes immobilized on contact lenses showed first-order release constants of 7.6 (±0.3)×10⁻³ h⁻¹ for 1 layer of liposomes, 2.4 (±0.5)×10⁻³ h⁻¹ for 2 layers, 1.5 (±0.6)×10⁻³ h⁻¹ for 5 layers and 3.4 (±1.6)×10⁻³ h⁻¹ for layers made out of liposome aggregates (FIG. 10). This finding suggests that the first layer of immobilized liposomes (the one exposed to the incubating solution) has a faster release rate than those underneath.

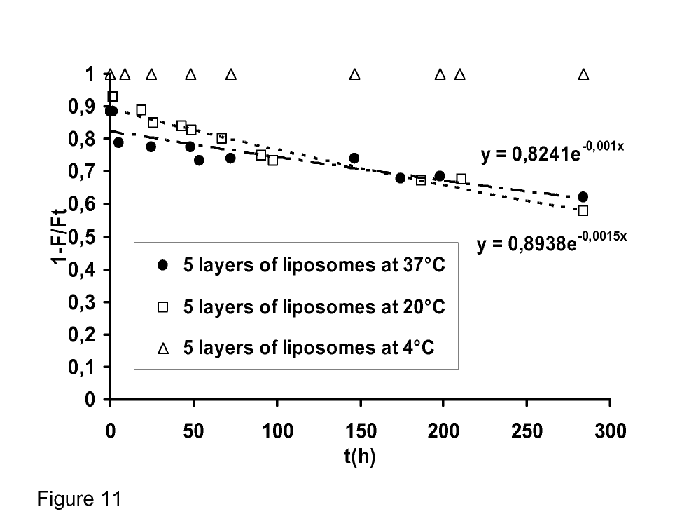

At 4° C. (FIG. 11), no CF release was observed from contact lenses bearing 5 layers of liposomes over 284 hours (ca. 12 days) and measurements of fluorescence carried out after one month show no further release. This finding shows that the liposome layers were stable at low temperature indicating that these lenses bearing layers of intact liposomes can be stored in a refrigerator with minimal lost of the liposome content.

At 37° C. (FIG. 11), 5 layers of liposomes immobilized onto contact lens surfaces showed a burst of release of approximately 18% during the first 4 hours and a first-order release constant of 1.0 (±0.5)×10⁻³ h⁻¹ for the next 280 hours. This also could suggest that the first layer of liposomes (i.e., that directly exposed to the solution) released the entrapped CF sooner, whereas the sub-layers are more stable.

On the basis of the concentration of the CF dye loaded into the liposomes (85 mM) and the surface area of a contact lens (ca. 5.6 cm²), the liposome density corresponds to approximately 80 immobilized liposomes per μm² or 0.8×10¹⁰ immobilized liposomes per cm² for 1 layer of liposomes, 300 immobilized liposomes per μm² or 3.0×10¹⁰ immobilized liposomes per cm² for 2 layers of liposomes and 860 immobilized liposomes per μm² or 8.6×10¹⁰ immobilized liposomes per cm² for 5 layers of liposomes. Given a liposome diameter of 106 nm, it is clear that this coverage is submonolayer for one layer of liposomes, and becomes a combination of several liposome layers for 2 and 5 layers of immobilized liposomes.

In view of the results obtained and without being bound to any particular theory, it is believed that 3D configuration provided by the liposome layers on which NeutrAvidin attach might favor a better subsequent NeutrAvidin attachment. The value calculated from the fluorescence measurements for 5 layers of liposomes seems to be in good agreement with the AFM images (FIGS. 6 to 9).

Conclusions

A strategy has been implemented and verified by XPS surface analyses for the immobilization of liposomes onto soft contact lenses of interest for the subsequent treatment of ocular diseases. The multilayer scheme utilized provides strong interfacial bonding, either covalent or by biotin/avidin affinity, between the individual layers, thus minimizing the risk that liposomes would detach from contact lenses surfaces. AFM data suggest that the liposome coverage consisted of several “sandwiched” layers of individual liposomes.

The release rate of carboxyfluorescein from CF-loaded liposomes was assessed and found to display a behavior indicative of diffusion control. No significant evidence was found for disruption of liposomes upon surface binding.

### Example 2

### Antibacterial Activity of Contact Lenses Bearing Surface-immobilized Layers of Intact Liposomes Loaded with Levofloxacin

In vitro methods for evaluation of antibacterial activity were used with soft contact lenses bearing levofloxacin loaded liposomes developed for the prevention and the treatment of bacterial ocular infection. Levofloxacin was incorporated into liposomes vesicles before these intact liposomes were immobilized onto the surface of soft contact lenses using a multilayer immobilization strategy. The release of levofloxacin from the contact lens into a saline buffer at 37° C. was monitored by fluorescence. The total release of levofloxacin from the contact lens was completed within 6 days. The antibacterial activity of the liposomes coated contact lenses against Staphylococcus aureus was evaluated by measuring the diameters of the inhibition zone on agar and the optical density of the broth. The liposomes coated contact lenses showed an antibacterial activity both on agar and in broth after 24 hours of incubation. The levofloxacin release study coincided with the observed antibacterial activity in broth.

The antibacterial activity of contact lenses bearing surface-immobilized layers of intact liposomes loaded with levofloxacin was investigated. Levofloxacin was chosen as a model drug for its broad antibacterial spectrum against both Gram-positive and -negative bacteria and its commercial availability. Staphylococcus aureus was selected to determine the antibacterial activity of the device since it is known to be a significant cause of the ocular infections. First, the kinetics release of levofloxacin from a liposomes coated contact lens was studied; then, the antibacterial activity was tested both on agar and in broth.

Materials Used for the Preparation of Surface Modified Contact Lenses

Contact lenses (Hioxifilcon B, Opti-Gel 45G, Opti-Centre, Sherbrooke, QC, Canada) were used as substrates for surface immobilization of intact liposomes immobilization.

Hexane (A.C.S. grade) was purchased from ACP (Montreal, QC, Canada).

Levofloxacin (98% purity, #28266), disuccimidylcarbonate (DSC, technical grade, #225827), anhydrous acetonitrile (CH₃CN, 99.9% purity, #271004), N-hydroxysuccinimide (NHS, #H-7377), N-[2-hydroxyethyl]piperazine-N′-[2-ethanesulfonic acid] (HEPES, #H-3375) (99.5%), t-octylphenoxypolyethoxyethanol (Triton X-100, #T-9284), 1-ethyl-3-(3-dimethylaminopropyl)carbodiimide (EDC, #E-1769), were purchased from Sigma-Aldrich (Oakville, ON, Canada).

Poly(ethylenimine) (PEI, 70 kDa MW, #00618) was obtained from Polysciences, Inc. (Warrington, Pa., USA).

Müeller-Hinton broth (MHB, #B11443), Tryptic soy broth (TSB, DF370173), agar (DF0054176), bacto agar (DF0140010), Brucelle broth, sodium chloride (NaCl) (A.C.S. grade), sodium sulfate (Na₂SO₄) (A.C.S. grade) and chloroform (A.C.S. grade) were purchased from Fisher Scientific (Ottawa, Ontario, Canada).

1,2-distearoyl-sn-glycero-3-phosphocholine (DSPC) (>99%, #850365), cholesterol (CHOL) (>99%, #700000), N-[ω-(biotinoylamino)poly(ethylene glycol) 2000]-1,2-distearoyl-sn-glycero-3-phosphoethanolamine (DSPE-PEG(2000)-Biotin) (>96%, #890129) were obtained from Avanti Polar Lipids Inc. (Alabaster, Ala., USA).

Biotin-PEG-CO₂—NHS (NHS-PEG-Biotin, #0H4M0F02) was purchased from Nektar Therapeutics (Huntsville, Ala., USA).

NeutrAvidin™ (ImmunoPure® NeutrAvidin™ biotin-binding protein, #31000) was obtained from Pierce (Rockford, Ill., USA). NeutrAvidin™ is a modified avidin with low non-specific binding properties and does not contain carbohydrate, thus eliminating the potential of binding to lectins (information obtained from Pierce, Rockford, Ill., USA).

S. aureus (ATCC 29213) were used in this study and grown aerobically at 37° C. on tryptic soy agar. Isolates were frozen at −80° C. in 1.5% Bacto agar and 25% Brucelle broth plus 15% glycerol, with two subcultures made before the organisms were tested.

Preparation of Liposomes

The solution of antibiotics used to prepare the liposome suspensions contained 100 mg/mL (270 mM/L) of levofloxacin at pH 7.4 and a NaCl concentration adjusted to an osmolarity of 290 mOsm using the Advanced™ osmometer, model 3250 (Advanced Instruments Inc., Norwood, Mass., USA). Milli-Q gradient water (Millipore Canada, Nepean, Ontario, Canada) with a resistivity of not less than 18.2 MΩ·cm was used to prepare the antibiotics solutions. The levofloxacin solution was filtered using sterile 0.22-μm filters (Millex® GP, Millipore, Cork, Ireland) before use in the preparation of the liposome suspensions.

Unilamellar vesicles (ULVs) were prepared by first mixing in a round-bottom flask DSPC, cholesterol, and DSPC-PEG(2000)-Biotin (2:1:5 mol % mol ratios) in HPLC-grade chloroform. Approximately 144 μmol of lipids were deposited from 2 ml HPLC-grade chloroform solution to form a thin film on the interior surface of a 50 mL round-bottom flask by rotary evaporation under a pressure of ˜13 332 Pa for 4 h. Following addition of levofloxacin solution, lipids were hydrated in the dark above 65° C. The multilamellar vesicle (MLV) suspension thus produced was subjected to ten freeze-thaw cycles involving quenching in dry ice and in acetone, followed by immersion in a 65° C. water-bath. Unilamellar vesicles were finally produced by extrusion through 100-nm pore polycarbonate Avestin® track-etch membranes using the Avestin Liposofast (Avestin Inc., Ottawa, Ontario, Canada) operated at 65° C.

Immobilization of Liposomes

Each contact lens was sonicated in 4 mL of anhydrous acetonitrile for 15 min to remove any trace of contaminants. Each contact lens was then immersed in 3 ml of a DSC solution for 1 hour under vigorous shaking. The activation of hydroxyl groups can also, if desired, be made by using either epoxides, oxiranes, oxidation with periodate, enzymatic oxidation, alkyl halogens, isocyanates, or carbonyldiimidazole (CDI). After this step, the lenses were rinsed in acetonitrile and immersed in a 3 mg/ml solution of polyethylenimine (PEI) in Milli-Q water with the pH adjusted to 7.4 with 1 M HCl. The reaction was allowed to proceed overnight under vigorous shaking. To remove any non-covalently adsorbed PEI, the contact lenses were then rinsed overnight under vigorous shaking in a 150 mM NaCl solution with the solution changed twice. The lenses were finally soaked overnight in Milli-Q water with the solution changed twice prior to further use.

PEI-coated lenses were immersed in a 1 mg/ml solution of NHS-PEG-Biotin under cloud point conditions (170 mg. ml⁻¹ of Na₂SO₄ were added to the solution of NHS-PEG-Biotin in order to form aggregates of PEG). 0.7 mg ml⁻¹ of EDC and NHS was added to the NHS-PEG-Biotin solution during the coupling procedure to re-activate the ester groups that could be potentially hydrolysed. The reaction was allowed to proceed overnight at room temperature under vigorous shaking. To remove any non-covalently attached NHS-PEG-Biotin, the contact lenses were then rinsed overnight under vigorous shaking in a 150 mM NaCl solution with the solution changed twice. The lenses were finally soaked overnight in Milli-Q water with the solution changed twice.

Next, PEG-Biotin-coated contact lenses were immersed in a 50 μg/ml solution of NeutrAvidin in 10 mM HEPES buffer at pH 7.4. The reaction was allowed to proceed overnight at room temperature. To remove any proteins not linked to PEG-biotin (i.e., NeutrAvidin molecules that can be loosely adsorbed onto the PEG layer), samples were rinsed overnight in a 10 mM HEPES solution with the solution changed twice.

Immobilization of liposomes was performed by incubating the NeutrAvidin-coated lenses in a 1 mg/ml (total lipid concentration) biotinylated-liposome suspension made of DSPC:CHOL:DSPE-PEG(2000)-Biotin (2:1:5 mol %) for 1 h. NeutrAvidin-coated contact lenses were immersed in 3 ml of the biotinylated-liposome suspension. To remove loosely adsorbed liposomes, the samples were rinsed for 1 hour in the HEPES buffer. The buffer solution was changed three times.

Multi-layers of liposomes were fabricated by adding, after the attachment of the first liposome layer, more NeutrAvidin, which can add to biotins on the solution side of the liposomes present, following which, more liposomes can be added to bind onto the NeutrAvidin molecules, and so forth. Contact lenses bearing one layer of surface-immobilized biotinylated liposomes were immersed in a 50 μg/ml solution of NeutrAvidin in 10 mM HEPES buffer at pH 7.4 for 30 minutes. They were rinsed for 1 hour in the HEPES buffer with the solution changed three times. A next layer of liposomes was then added to the surfaces by incubating them in the 1 mg/ml biotinylated-liposome suspension.

Contact lenses with and without levofloxacin loaded liposomes were sterilized by a 5 min soaking of ethanol 70% just before the liposomes attachment, the next steps were done in a laminar flux hood in sterile conditions.

In Vitro Levofloxacin Release

Liposomes containing levofloxacin were immobilized onto NeutrAvidin™-coated contact lenses and the release of levofloxacin from liposomes was monitored over time using a Bio-Tek Synergy HT well-plates reader (Bio-Tek. Instruments, Winooski, VE, USA). Then, 3 ml of the appropriate medium (either 0.5% w/v Triton X-100 solution made with Milli-Q water or saline buffer (NaCl 150 nM at pH=7.4)) was added to each vial containing a contact lens bearing immobilized liposome layers and incubated at 37 C. At low concentration and neutral pH, Triton X-100 instantaneously disrupts the vesicles and liberates their contents without significant interference with the intrinsic fluorescence of the levofloxacin. At periodic intervals, 200 μL of the incubating solution was withdrawn from the vial containing the lenses and transferred into 96-well plates for fluorescence measurement. After the fluorescence measurement, the 200 μL incubating solution withdrawn for the fluorescence measurement were returned to the vial containing the lens and mixed thoroughly. The fluorescence signal was monitored at 460 nm (excitation at 310 nm and emission at 460 nm). Each experiment was done in triplicate.

The levofloxacin release over time was calculated using Equation 1:
Fraction of levofloxacin remaining in vesicles=1−F/F_{T} (1)
where F is the fluorescence at 460 nm measured at any time during the experiment, and F_{T} is the total levofloxacin fluorescence at 460 nm determined after disruption of the vesicles with Triton X-100.

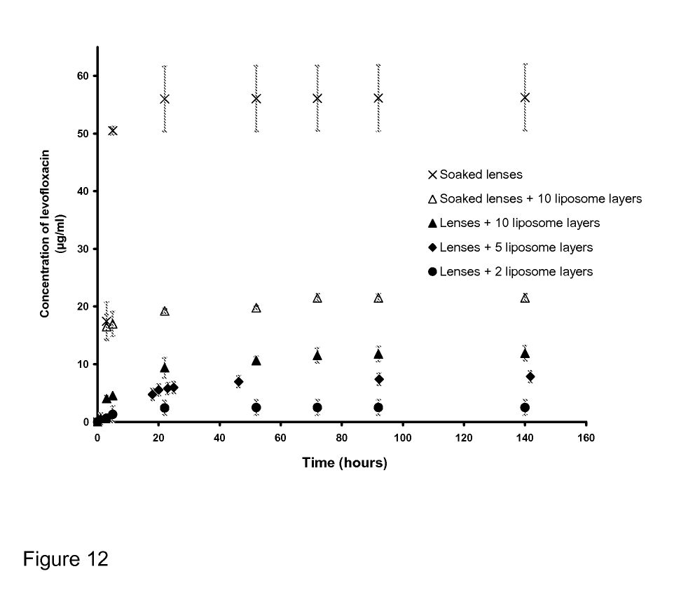

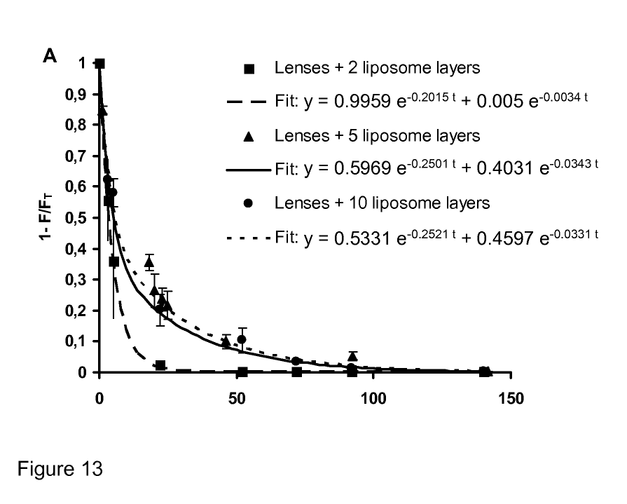

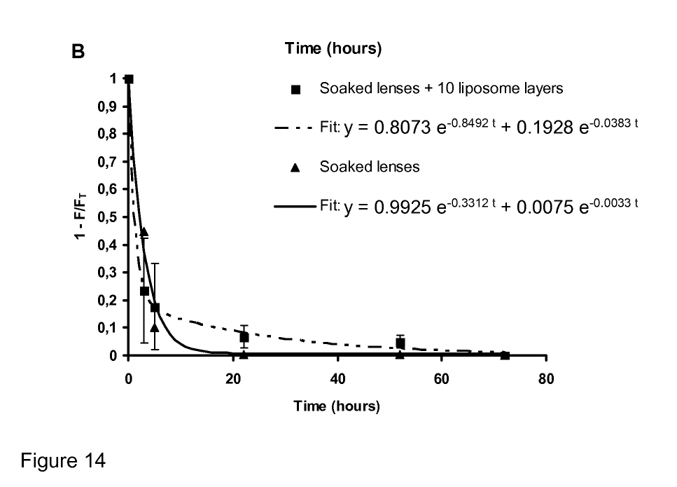

The release kinetics of levofloxacin from contact lenses coated with layers of stable liposomes loaded with levofloxacin is shown in FIGS. 12 to 14. On the molecular scale, the course of the release of levofloxacin may be complex, but the form of the empirical rate law shown in FIGS. 13 and 14 suggests that the particular path via which the release of levofloxacin takes place follows first order release models. Overall, this suggests that levofloxacin was released by diffusion rather than disruption of the liposomes. To compare the release kinetics of each lens, the collected data were analyzed by exponential functions.

FIGS. 13 and 14 show the experimental data as well as the mathematical models correlating the progression of the levofloxacin release from the different modified lenses. The experimental data depicted in FIGS. 13 and 14 were in good agreement with the nonlinear correlations shown in these same figures and solved by the Nelder-Mead simplex algorithm. A clear tendency was exhibited by the levofloxacin release. The overall levofloxacin release from contact lenses bearing layers of stable liposomes appeared to be a combination of the following mechanisms represented by two-independent first-order kinetics.

TABLE 3
Overall levofloxacin release from contact lenses bearing layers of stable liposomes
can be modelled by a combination of two-independent first-order kinetics.

| | 1/Kc (hours) | | |
|---|---|---|---|
| | time to release 63.2% of the total | | % of the total liposome- |
| Modified lenses | liposome-encapsulated levofloxacin | | encapsulated levofloxacin |
| Lenses + | 1^{st} first-order model: | 4.96 | 99.59 |
| 2 layers of liposomes | 2^{nd} first-order model: | 294.1 | 0.41 |
| Lenses + | 1^{st} first-order model: | 3.99 | 59.69 |
| 5 layers of liposomes | 2^{nd} first-order model: | 29.2 | 40.31 |
| Lenses + | 1^{st} first-order model: | 3.97 | 53.31 |
| 10 layers of liposomes | 2^{nd} first-order model: | 30.2 | 45.97 |
| Soaked lenses + | 1^{st} first-order model: | 1.2 | 80.73 |
| 10 layers of liposomes | 2^{nd} first-order model: | 26.1 | 19.27 |
| Soaked lenses | 1^{st} first-order model: | 3.02 | 99.25 |
| | 2^{nd} first-order model: | 303.03 | 0.75 |

Firstly, following incubation in the buffer solution, the amount of levofloxacin that transferred from the layers of liposomes to the buffer solution exhibited a fast release rate. For lenses bearing two layers of liposomes, this rapid, dynamic behaviour (as illustrated by the first term of the fits shown in FIGS. 13 and 14) was associated to a mass transport phenomenon with a mass transfer coefficient (Kc) of 0.2015 hour⁻¹, which can be viewed as a first-order system with a time constant (1/K_{c}) of 4.96 hours. This time constant corresponds to the time of incubation of the modified lenses in the buffer solution to release 63.2% of 99.59% of the total liposome-encapsulated levofloxacin. This first term clearly shows that most levofloxacin encapsulated in the immobilized liposomes is released fairly rapidly. For lenses bearing 5 and 10 layers of liposomes, the first terms of the fits shown in FIGS. 13 and 14 corresponding to a fast release were similar and were associated to mass transfer coefficients (K_{c}) of 0.2501 hour⁻¹ and 0.2521 hour⁻¹, respectively, which can be viewed as first-order systems with time constants (1/K_{c}) of 3.99 and 3.97 hours, respectively. These time constants correspond to the time of incubation of the modified lenses in the buffer solution to release 63.2% of 59.69% and 53.31% of the total liposome-encapsulated levofloxacin, respectively, for these two systems. This analysis revealed that the contribution of the fast release rate of levofloxacin over the total release of levofloxacin was smaller for lenses bearing 5 and 10 liposome layers than for those bearing only 2 layers.

For contact lenses bearing 10 layers of liposomes and those bearing no liposome both soaked overnight in a solution of levofloxacin at 5 mg/ml, the first terms of the fits shown in FIGS. 13 and 14 associated with a fast release rate corresponded to mass transfer coefficients (K_{c}) of 0.8492 and 0.3312 hour⁻¹, respectively, which can be viewed as first-order systems with time constants (1/K_{c}) of 1.2 and 3 hours, respectively. These time constants also correspond to the time of incubation of the lenses in the buffer solution to release 63.2% of 80.73% and 99.25% of the total loaded levofloxacin, respectively, for these two lenses. Although these lenses can load larger amount of levofloxacin than non-soaked lenses bearing only liposome layers (see FIG. 12), this analysis reveals that soaked contact lenses (with or without liposome layers) show faster release of their total loaded levofloxacin than non-soaked lenses bearing only liposome layers. Lenses bearing no liposome and soaked in a levofloxacin solution show a burst release, corresponding to more than 99% of their total loaded medication in ca. 3 hours.

Secondly, following this initial fast release rate, a second and much slower levofloxacin release took place with the lenses bearing 5 and 10 liposome layers. This slower dynamics was well correlated by the second terms of the fits presented in FIGS. 13 and 14. The mass transfer coefficients determined for the second terms of the fits for lenses bearing 5 and 10 liposome layers were in fact equivalent to a transport process with time constants of 29.2 and 30.2 hours, respectively. The mass transfer coefficient determined for the second term of the fit for lenses bearing 10 liposome layers and subsequently soaked in a levofloxacin solution was equivalent to a transport process with a time constant of 26.1 hours. As shown in FIGS. 13 and 14, the response of the second term was slower, as the release of levofloxacin increased very slowly over time. FIGS. 13 and 14 also clearly shows that the progression of the levofloxacin release over time expressed by the first term of the fits overtakes the progression of the second-term for the levofloxacin-soaked lenses and for the lenses bearing 2 layers of levofloxacin-loaded liposomes. Nevertheless, by combining the two models, the dynamics of the overall nonlinear correlation perfectly matched that of the experimental data monitored by fluorescence measurements.

In summary, the mechanisms that drive levofloxacin release as a function of the time of incubation in a buffer solution at 37° C. were shown to be divided into two steps. First, considering the direct exposure of the outermost liposome layer, the levofloxacin release was almost instantaneous, and almost completed following a short period of time for the lenses bearing 2 layers of liposomes and the soaked lenses bearing no liposome. This assumption was supported by the small time constant obtained from the first term of the nonlinear mathematical model. Upon addition of more liposome layers on the surfaces of the contact lenses, it can hypothesized that these layers of liposomes became a de facto filter medium for the diffusing levofloxacin. The deposited liposomes in fact created an additional resistance to the drug diffusion. The liposome layers can be seen as a bulky mass of vesicles, among which appeared to run small channels that allowed a restrictive molecular movement. This behaviour was supported by the large time constants shown in the second term of the empirical correlation. Upon addition of more liposome layers, not only the total amount of levofloxacin was increased but the macromolecular mobility across the liposome layers became increasingly limited. This result in an increase contribution of the slower release rate over that of the faster release rate in the overall release of levofloxacin in function of the incubation time.

In the above study on carboxyfluorescein release from 5 layers of liposomes immobilized on contact lenses (Example 1), the release rate of carboxyfluorescein at 37° C. was lower than that found here. In fact, the concentration of levofloxacin used here (270 mM) is 3 times greater than the concentration of carboxyfluorescein used above (85 mM): this could explain why the diffusion is faster for the levofloxacin (larger concentration gradient across the liposomal membrane). Moreover, the physicochemical properties are different for the 2 fluorescent molecules: levofloxacin is 3 times more soluble than carboxyfluorescein.

The total amount of levofloxacin released from a contact lens (FIG. 12) was estimated to be respectively 8 (±3) μg (2.5 μg/ml in 3 ml of incubating solution) for a contact lens bearing 2 layers of liposomes, 24 (±1) μg for a contact lens bearing 5 layers of liposomes and 40 (±10) μg for a contact lens bearing 10 layers of liposomes, that corresponds to 45 times the required concentration to inhibit the growth of 106 colony forming units (CFU)/ml of Staphylococcus aureus ATCC 29213. (North D S, et al. 1998. Pharmacotherapy 18:915-935; Noviello S, et al. 2003., Journal of Antimicrobial Chemotherapy 52:869-872.)

From these results, it appears that contact lenses bearing 5 or 10 layers of liposomes can maintain a sustained delivery of levofloxacin until 120 hours, while the release from contact lenses bearing 2 layers of liposomes is completed in 30 hours. However, the total amount of loaded antibiotics could be insufficient if the bacteria are exceeding 50×10⁶ CFU/ml.

Contact lenses bearing 10 layers of liposomes soaked overnight in the antibiotic solution could be used to provide an initial burst release of levofloxacin during the first hours of infection followed by a sustained release from the surface-bound liposomes.

Assay of Antibacterial Activity

Antibacterial activity of levofloxacin loaded liposomes covalently bound onto contact lens against Staphylococcus aureus (ATCC 29213) was determined using both agar and broth.

In studies using the agar, the antibacterial activity of the modified contact lenses was assessed by a diffusion test on Müeller-Hinton agar culture plates (100 mm in diameter, 15 mm height, Fisher). Before the tests, 5 mm diameter discs samples were cut into each tested contact lens. The bacterial inoculum was prepared according to NCCLS standards (Kirby-Bauer). A sterile cotton swab was dipped into the bacterial inoculum broth suspension and excess fluid was removed by rotating the swab several times against the wall of the vessel. The inoculum was streaked evenly in 3 planes onto the surface of the agar. Then, one 5 mm diameter disc sample bearing levofloxacin loaded liposomes was placed at the centre of the right part of each plate and gently pressed down to ensure contact. The same procedure was employed with one 5 mm diameter disc control of a contact lens without liposomes: this one was placed at the centre of the left part of the plate. The diameters of the inhibition zone were measured after incubating the plates for 24 h at 37° C.

In studies using broth, a contact lens bearing liposomes loaded with levofloxacin was immersed into a Mueller-Hinton broth (3 mL) in a culture tube inoculated with 1.5 10⁸ CFU/mL. Then, it was placed in a 37° C. incubator for 6 h. The initial optical density of each tube was around 0.070 at 650 nm. The antibacterial activity of levofloxacin released from the contact lenses was determined by measuring the optical density of the broth using a spectrophotometer (Novaspec II, Pharmacia Biotech, Cambridge, England) at 650 nm, while that of clear broth was used as a blank. A positive control with S. Aureus (1.5 10⁸ CFU/mL) inoculated in the broth without contact lens was used.

Agar Method

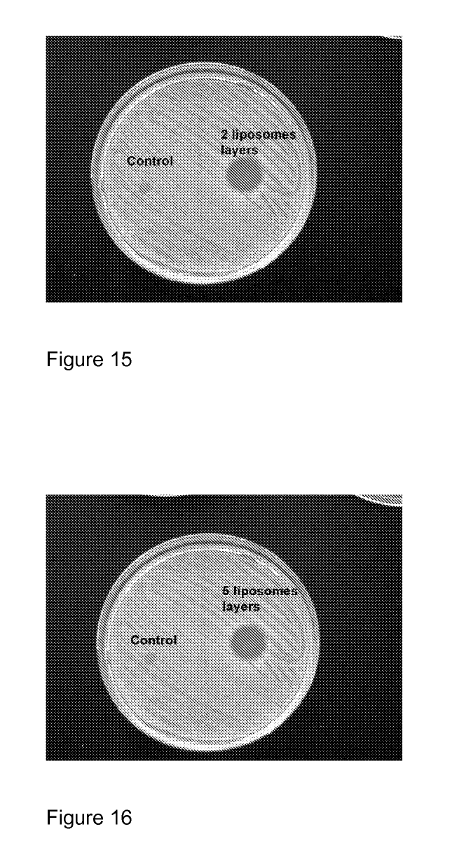

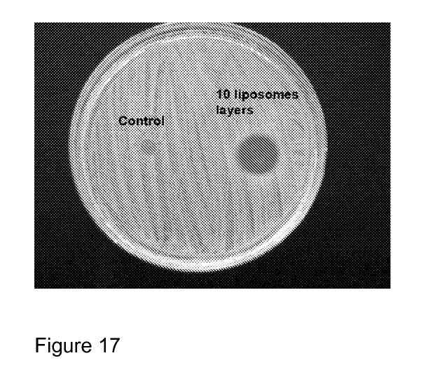

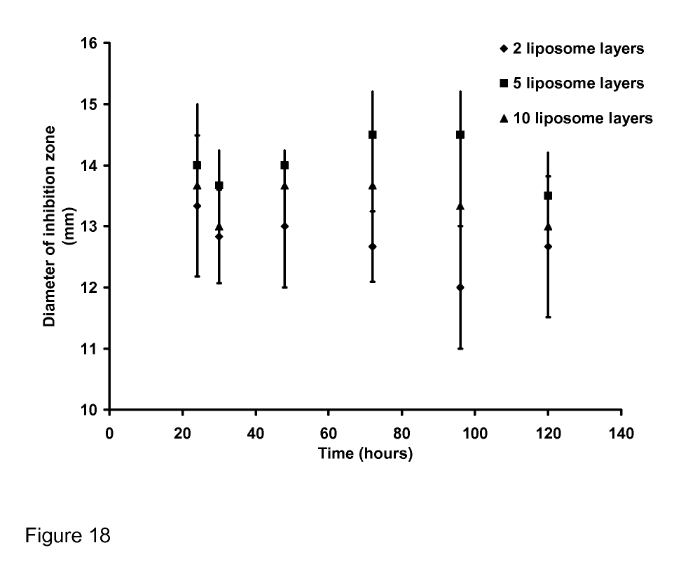

The antibacterial activity of liposomes coated contact lenses, determined using agar, against S. aureus is shown in FIGS. 15 to 18. All contact lens samples produced a zone of inhibition when placed in plates overlaid with S. aureus, while the control contact lens sample without levofloxacin loaded liposomes showed no inhibition zone (FIGS. 15 to 17). After 24 h incubation, the diameter of inhibition is quite similar for the 3 samples: 2, 5 and 10 liposomes layers (FIGS. 15, 16 and 17 respectively). For the next 5 days (FIGS. 15 to 18), the diameter of inhibition remains almost constant for the 3 samples. This suggests that the levofloxacin release was the same for the 3 samples for 5 days: just the first layers of liposomes have released levofloxacin on the agar whereas in the release assay in solution the complete release is obtained within 140 hours (6 days). Without being bound to a particular theory, it may be that the release and the diffusion of levofloxacin into the solution were easier than into the agar (solid state).

Broth Method

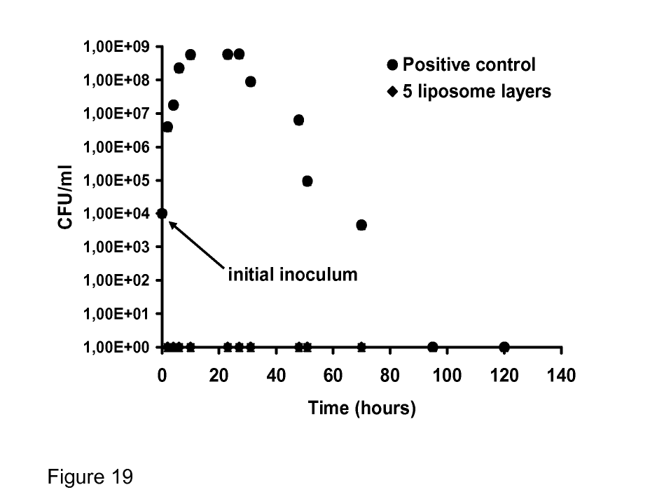

The antibacterial activity of the contact lenses bearing layers of liposomes loaded with levofloxacin was also investigated in broth. Due to their low-loading capacity, the contact lenses with 2 layers of liposomes were not used for this test. Three different concentrations of S. aureus ATCC 29213 (10⁴, 10⁶ and 10⁸ CFU/ml) were inoculated to compare the antibacterial effect of the samples. The results are reported in FIGS. 19 to 22.

FIG. 19 shows the antibacterial activity obtained with an initial inoculum of 10⁴ CFU/ml. All the tested contact lenses exhibited a complete inhibition of the S. aureus growth, whereas the positive control (a contact lens without any treatment immersed into the broth with the inoculum) shows an exponential growth followed by stationary and death phases due to the lack of nutrients in the broth after 24 hours.

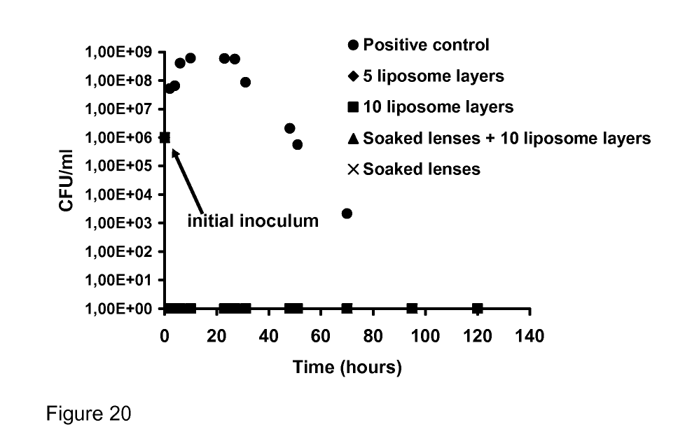

A similar trend was observed in FIG. 20 with an initial inoculum of 10⁶ CFU/ml: all the treated contact lenses inhibit the inoculated S. aureus within 2 hours.

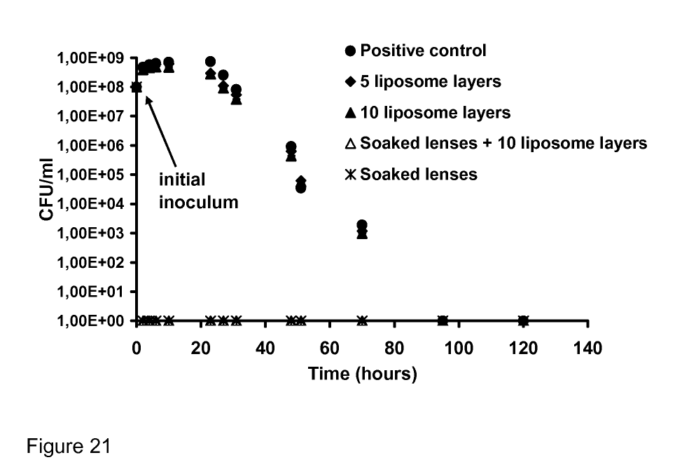

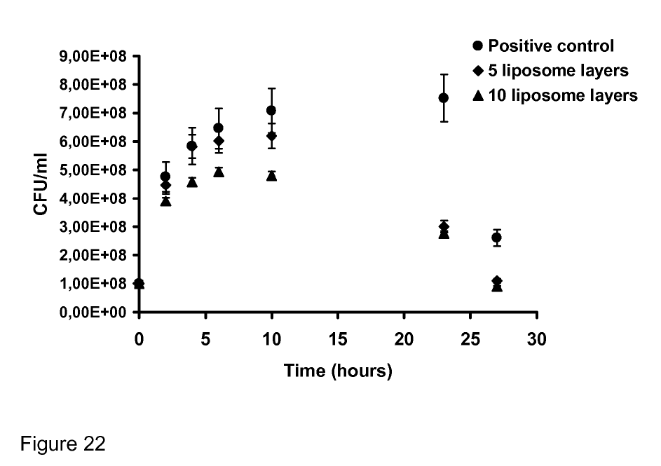

When the bacteria inoculum was higher i.e., 10⁸ CFU/ml (FIG. 21), only the soaked-contact lenses (those bearing 10 layers of liposomes and those re-hydrated) demonstrated a complete inhibition within 2 hours. From FIG. 22, which is the enlarged left part of FIG. 21, it can be seen that the lenses bearing 5 and 10 layers of liposomes progressively developed an antibacterial activity compared to the growth observed in positive controls. The contact lenses bearing 10 layers of liposomes loaded with levofloxacin reveal an antibacterial effect after 4 hours and lead to an inhibition of 32% of the S. aureus at 10 hours. For the contact lenses bearing 5 layers of liposomes, the antibacterial effect begins after 6 hours and reaches 12% of inhibition at 10 hours.

These results are in good agreement with the levofloxacin release observed with the kinetics results. In fact, the amount of levofloxacin released from the contact lenses bearing 5 and 10 layers of liposomes loaded with levofloxacin was evaluated to be sufficient to inhibit bacteria growth for initial bacterial inocula of 50×10⁶ CFU/ml. This explains why all the bacteria were inhibited within 2 hours when the inocula did not exceed 10⁶ CFU/ml and why there was a lower antibacterial efficiency when the bacteria inoculum was 10⁸ CFU/ml. Then, when the inoculum was between 50×10⁶ and 10⁸ CFU/ml, the soaked contact lenses are advantageous due to their initial higher release capacity: this is demonstrated in FIG. 21.

In the case of a keratitis due to S. aureus in a rabbit model, the number of bacteria in a rabbit cornea 10 hours post-infection was reported to be approximately 10⁷ CFU. (Oguz H, et al., 2005. Current Eye Res 30:155-161.). Thus, soaked-contact lenses bearing 10 layers of liposomes loaded with levofloxacin seem to have the advantage to combine a burst release used to inhibit a large amount of bacteria that can be present at the beginning of the infection and necessary to stop the fast exponentially growing bacteria, followed by a sustained release to complete the antibacterial effect.

Each study was performed in triplicate.

Conclusions

These examples demonstrate that contact lenses bearing surface-immobilized layers of intact liposomes loaded with levofloxacin can provide a sustained release of antibiotics over 6 days. The contact lenses coated with levofloxacin loaded liposomes show an antibacterial activity against Staphylococcus aureus after 24 hours of incubation on agar and in broth.

### Example 3

### Biocompatibility and Transmission Spectra of Contact Lenses Bearing Surface-Immobilized Layers of Intact Liposomes

The biocompatibility of soft contact lenses coated with liposomes was evaluated through in vitro direct and indirect cytocompatibility assays. The investigations were performed with epithelial cells from human cornea cultured in monolayers, on reconstructed human corneas, and on ex vivo rabbit corneas since the liposomes attached to the lens will be in direct contact with the external surface of the cornea. Transmittance spectra of these liposome-covered contact lenses were also measured to test whether or not they fulfill their optical function with a minimum of light dispersion or color alteration.

Materials

Contact lenses (Hioxifilcon B, Opti-Gel 45G, Opti-Centre, Sherbrooke, QC, Canada) were used as substrates for surface immobilization of intact liposomes. All similar lenses had the following parameters: power: −3.00D; total diameter: 14.50 mm; base curve: 8.60 mm; brand: 51% HioxifilconB+49% water. The elemental composition of the contact lens surface has been reported above by X-ray photoelectron spectroscopy (XPS) and is typical of the two polymers composing the hioxifilcon material: (i) poly(2-hydroxyethylmetacrylate) [p(HEMA)] and (ii) polyglycidylmetacrylate [p(GMA)].

Hexane (ACS grade) was purchased from ACP (Montreal, QC, Canada). Disuccimidylcarbonate (DSC, technical grade, #225827), anhydrous acetonitrile (CH₃CN, 99.9% purity, #271004), N-hydroxysuccinimide (NHS, #H-7377), N-[2-hydroxyethyl]piperazine-N′-[2-ethanesulfonic acid] (HEPES, #H-3375, 99.5%), 1-ethyl-3-(3-dimethylaminopropyl)carbodiimide (EDC, #E-1769) were purchased from Sigma-Aldrich (Oakville, ON, Canada). Poly(ethylenimine) (PEI, 70 kDa MW, #00618) was obtained from Polysciences Inc. (Warrington, Pa., USA). Sodium chloride (NaCl, ACS grade), sodium sulfate (Na₂SO₄, ACS grade) and chloroform (HPLC-grade) were purchased from Fisher Scientific (Ottawa, Ontario, Canada). 1,2-distearoyl-sn-glycero-3-phosphocholine (DSPC, >99%, #850365), cholesterol (CHOL, >99%, #700000), N-[ω-(biotinoylamino)poly(ethylene glycol) 2000]-1,2-distearoyl-sn-glycero-3-phosphoethanolamine (DSPE-PEG(2000)-Biotin, >96%, #890129) were obtained from Avanti Polar Lipids Inc. (Alabaster, Ala., USA). Biotin-PEG-NHS (NHS-PEG-Biotin, #0H4M0F02) was purchased from Nektar Therapeutics (Huntsville, Ala., USA). NeutrAvidin (ImmunoPure® NeutrAvidin™ biotin-binding protein, #P31000) and D-biotin (ImmunoPure® D-Biotin, #29129) were obtained from Pierce (Rockford, Ill., USA).

Liposome Preparation and Immobilization

Detailed procedures used to produce liposomes and to immobilize these intact liposomes on contact lens surfaces are described above. Briefly, 100-nm unilamellar vesicles (Hope M, et al. Chem Phys Lipids 1986; 40:89-107) were made using DSPC, cholesterol, and DSPC-PEG(2000)-Biotin (2:1:(5 mol % of the total lipid content)) by extrusion through 100-nm pore polycarbonate Avestin® track-etch membranes using the Avestin Liposofast (Avestin Inc., Ottawa, Ontario, Canada). Contact lenses were firstly activated in a DSC solution made in anhydrous acetonitrile and then immersed in a solution of polyethylenimine (PEI) in Milli-Q water with the pH adjusted to 7.4. PEI-coated lenses were immersed in a 1 mg/ml NHS-PEG-Biotin solution under cloud point conditions (170 mg/ml of Na₂SO₄ were added to the NHS-PEG-Biotin solution to form aggregates of PEG). Under these conditions, the resulting PEG layers exhibited excellent low-fouling properties. The next step was the immersion of PEG-Biotin-coated contact lenses in a 50 μg/ml NeutrAvidin solution of 10 mM HEPES buffer at pH 7.4 (Vermette P, et al. Journal of Colloid and Interface Science 2003; 259:13-26). Immobilization of liposomes was performed by incubating the NeutrAvidin-coated lenses in a 1 mg/ml (total lipid concentration) biotinylated-liposome suspension made of DSPC:CHOL:DSPE-PEG(2000)-Biotin (2:1:(5 mol % of the total lipid content)) for 1 hour. Multi-layers of liposomes were fabricated by adding, after the attachment of the first liposome layer, more NeutrAvidin, which can add to biotins on the solution side of the liposomes present, following which, more liposomes can be added to bind onto the NeutrAvidin molecules, and so forth. Lenses were thoroughly rinsed between each modification step.

Contact lenses were sterilized by a 5-min soaking in 70% ethanol (HPLC-grade) just before liposome attachment and the next steps were done in a laminar flow cabinet under sterile conditions. This method was sufficient to sterilise the lenses as no contamination was found. Ethanol-incubated lenses were thoroughly rinsed overnight in sterile Milli-Q water with the water solution changed several times to remove any trace of ethanol.

Cell Culture Studies

For all procedures in cell cultures, corneal epithelial and stromal cells were isolated from post-mortem human corneas following a method previously reported. (Griffith M, Osborne R, Munger R, Xiong X, Doillon C J, Laycock N L, Hakim M, Song Y, Watsky M A. Functional human corneal equivalents constructed from cell lines. Science 1999; 286(5447):2169-2172.) Cells were grown in Dulbecco's modified Eagle's medium (DMEM, Sigma-Aldrich Canada Ltd.) with 10% fetal bovine serum (FBS; Qualified US/Invitrogen Co., Burlington, ON) in the presence of L-glutamine (2 mM) and 1% Insulin-Transferrin-Selenium-A (ITS; Invitrogen Co.). In the quantitative measurements, data were compared by the ANOVA tests for significant difference with a p-value set at 0.05. Cell culture assays with or without serum were used to investigate whether or not the presence of serum proteins affects the interactions of cells with the liposome-modified lenses. For example, protein adsorption may affect cell response towards biomaterials like the lens in direct contact with epithelial cells.

In Vitro Direct Contact Biocompatibility Assay

The possibility of a cytotoxicity effect of liposome-bearing lenses was verified in direct contact with epithelial cells in cultures. Human corneal epithelial cells were seeded at 1×10⁵ cells on each well of 24-multiwell plates to reach sub-confluence. The viability of the cells was then measured by their mitochondrial activity using an in vitro proliferation assay kit (XTT assay or TOX2, Sigma-Aldrich Canada Ltd). The XTT (2,3-bis{2methoxy-4-nitro-5-sulfophenyl}-2H-tetrazolium-5-carboxyanilide inner salt) assay was performed according to the manufacturer's procedures. Lenses bearing stable liposomes and those without liposome (control) were directly laid on the monolayer of epithelial cells for a 24-hour period in serum-free medium. A PBS solution of XTT (1 mg/ml) was mixed with a PBS solution of phenazinemethosulfate and incubated at 37° C. for 1 hour. The absorbance was spectrophometrically measured at a wavelength of 450 nm.

Epithelial Cell Growth Assay

To further investigate the cell response towards lenses in direct contact with epithelial cells, a cell growth assay was performed. A density of 1×10⁴ cornea epithelial cells was seeded on each well of 24-multiwell plates. After 1 day of culture in serum-supplemented media, the lenses bearing or not liposomes were laid on the epithelial layer and incubated with cells for a 48-hour period in serum-free medium. After this culture period, cells were lyzed in a trisodium citrate solution in the presence of 0.02% SDS for 1 hour and reacted with Hoechst 33258 (0.5 μg/ml, Invitrogen Co). Fluorescence was then quantified in fluorocytometric plates using a BioTek FL-600 fluorometer (excitation 365 nm/emission 450 nm).

Elution Assay

To investigate the effect of a potential release of leaked compounds from the lenses, the following indirect assay was performed. Lenses were incubated for specific periods in culture media that were then used to grow epithelial cells. The different lenses were incubated at 37° C. in a 5% serum-supplemented DMEM medium for 3 and 10 days. The respective media were then tested on a monolayer of epithelial cells in culture for a 24-hour period following the same procedure as described above. The XTT assay kit was used to assess the cell viability.

Reconstruction of Bi-Layered Human Corneas

Since the stroma in the cornea and the Bowman's membrane may influence the organization of cornea epithelial cell lining, a reconstructed bi-layered cornea was reproduced on which the lenses were laid directly on the epithelial cell layer. Reconstruction of stroma layer was first performed according to a previously reported method. (Griffith M, et al. Science 1999; 286(5447):2169-2172.) Briefly, stromal cells were mixed in collagen gel (from rat tail tendon) at density of 5.0×10⁴ cells per 500 μl of gel in each well of 24-multiwell plates, and a gel was formed at neutral pH and in a culture incubator. A reconstituted Bowman's membrane was laid on the gel and corneal epithelial cells were seeded on the membrane at a density of 1.0×10⁵ cells per ml. Reconstituted cornea were incubated overnight. Lenses bearing liposomes and those without liposome (control) were set on the epithelium formed on the surface of the cornea construct. Cell cultures were incubated for 3 days in an incubator under 5% CO₂ in water saturation at 37° C. After fixation with formaldehyde, and paraffin embedding, histological sections were processed transversally and stained for hematoxylin, phloxin and saffron (HPS).

Ex Vivo Study on Rabbit Corneas

An ex vivo model was used to verify the epithelial cell response according to the physiological curvature of the cornea (biomechanical effect) on which the lens fitted on its whole surface. The cornea equivalent was rather a flat surface towards the lens compared to the ex vivo model. Corneas with the surrounding sclera were extracted, with great care to minimize damages to the corneas, from rabbit eyes obtained from a local abattoir. Corneas/scleras were sterilized by successively soaking them in an antibiotic solution made in phenol-free Hanks' balanced salt solution (Sigma) with Penicillin-Streptomycin-Neomycin (1X, Invitrogen Co.) and in a solution of 50 μg/ml gentamicin (Invitrogen Co.). The contact lenses were laid on a cornea/sclera and left floating in culture medium of wells of 6-multiwell plates for a 48-hour period. After removal of the lenses, an ophthalmic carboxyfluorescein solution was laid on the corneal surface and observed under fluorescence microscope. Corneas were then fixed with formaldehyde and the epithelium was colored by a toluidine blue solution (0.1%) for morphological observations. Histological sections were processed through corneas and stained for HPS.

Ultraviolet-Visible Transmission Assays

Transmission spectra of liposome-covered lenses were measured in the UV-visible wavebands for the following reasons. First, to fulfill their optical function without a gross alteration of color perception, it is preferred that contact lenses transmit more or less equally all wavelengths of the visible spectrum. Secondly, as reviewed recently, (Giasson C J, et al. Int Opthalmol Clin 2005; 45(1):117-139.) the eyes of contact lens wearers benefit from the protection afforded by an ultraviolet absorber included into the lens polymer.

The use of an integrating sphere during the measurement of the transmission spectrum, as recommended by the ISO and ANSI specifications, allows to measure directly transmitted light as well as of the forward-scattered light. This last portion of light is then measured, and not erroneously calculated as absorbed (Giasson C J, et al., supra; Faubl H and Quinn M H. Int Contact Lens Clin 1998; 25 (September-October 1998):142-148; Chou B R, et al., Int Contact Lens Clin 1988; 15(8):244-250.) The use of an integrating sphere yields sometimes, but not always, to a difference in the estimated absorption. These standards ensure that the light scattered by the contact lens reaches the detector of the integrating chamber and is not falsely considered as absorbed in Beer's law (ANSI, American National Standard for Ophthalmics—Contact lenses—standard terminology, Tolerances, Measurements and Physicochemical Properties ANSI Z80.20-1998. 1998, ANSI: New York. p. i-62. Krug H, Leitner E. (International Organisation for standardization (ISO), Geneva, Switzerland.). Optique et instruments d'optique—Lentilles de contact—Détermination du facteur spectral de transmission et du facteur relatif de transmission dans le visible. Report ISO 8599; 1994, Dec. 5).

The direct and indirect transmission spectra of contact lenses were measured with a double beam Varian UV-Vis-IR (Cary 5000) spectrophotometer controlled by the Cary WinUV 3.0 software (Mulgrave, Australia). Indirect transmission spectra used the integrating sphere (radius of 110 mm) of the internal diffuse reflectance accessory (DRA-2500). A pupil (diameter of 6 mm) was placed in front of the measuring cell for all measurements, which were carried out at 22° C. A 100%-0% baseline was measured before proceeding to any measurement of transmission spectrum of contact lenses. A quartz measuring chamber, (Quesnel N M, et al. Optom V is Sci 1995; 72(1):2-10.) filled with saline solution and centered in front of either the measurement beam (direct transmission) or of the absorption port of an integrating sphere (indirect transmission), was scanned (2 nm bandwidth, 600 nm/min) from 220 to 800 nm while the 100% baseline transmittance was measured. A similar second measurement was made except that a black cardboard was placed in the optical path of the instrument (0% baseline transmission). Then, a contact lens, removed from its vials with clean tweezers, was positioned in the measuring chamber and transmission spectra were measured and corrected for baseline. Spectra were measured three times for each lens.

Transmittance values at each scanning wavelength were used to calculate mean transmittance as well as the standard deviation (SD) for each wavelength. The repeatability, assessed by the SD of three measures, varied between 0.08% and 0.28% for direct measurements and between 0.42 and 0.73% for indirect transmittance. Differences in transmittance between the lens types were tested for significance at 280 and 550 nm with paired t-tests between each type of lenses, using the SPSS 13.0 software for Windows (Chicago, Ill.).

Direct Biocompatibility Assay

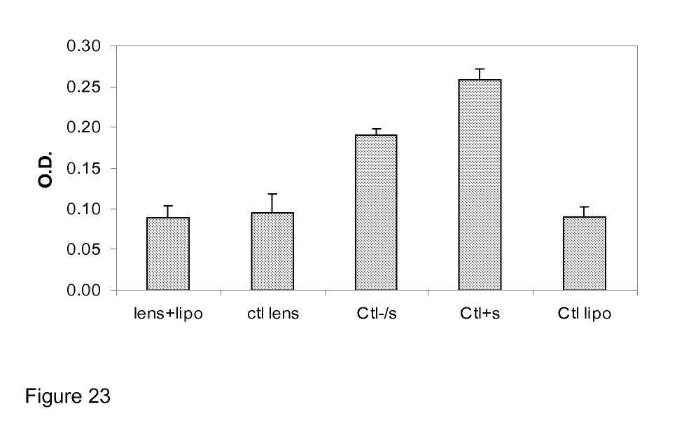

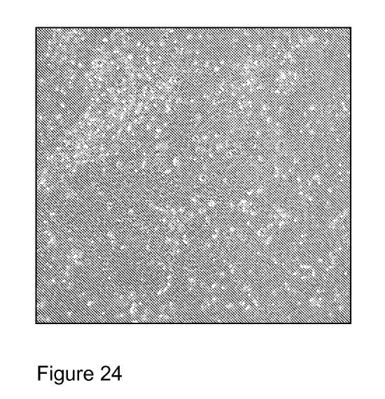

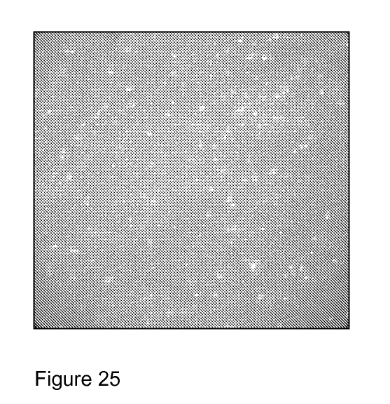

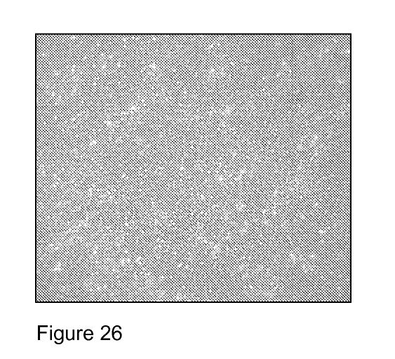

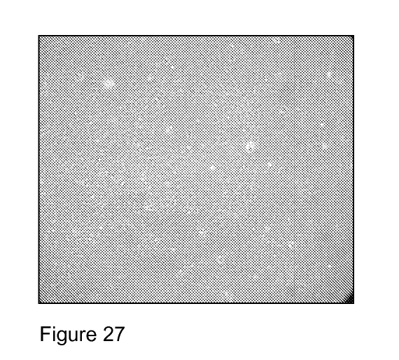

Screening for any cytotoxicity of modified contact lenses and the presence of liposomes was performed. Contact lenses bearing five layers of stable liposomes did not induce any significant changes in cell viability of human cornea epithelial cell layers compared after 24-hour incubation with control lenses bearing no liposome or with liposome suspensions (FIGS. 23 to 27). Conversely the control cultures with or without serum exhibited higher cell viability by 24 hours. This is probably due to a decreased of medium density caused by the close contact between the cells and the contact lenses. However, the epithelial cell monolayer appeared normal in any culture conditions (FIGS. 24 to 27). These results clearly demonstrate that the addition of liposome layers on the contact lens surfaces does not result in a decrease of corneal epithelial cell viability when compared to bare contact lenses.

Cell Growth Assays

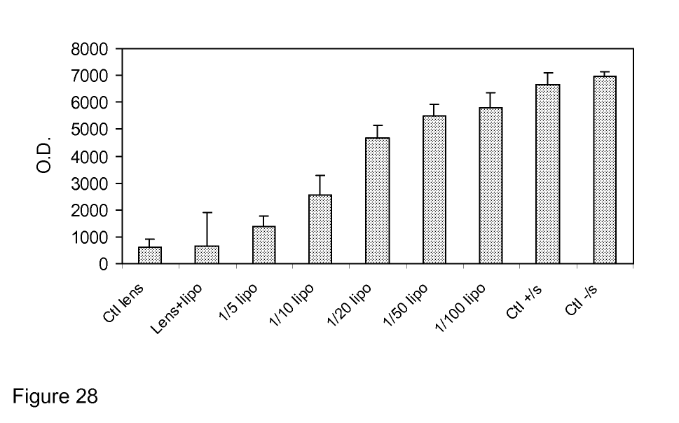

The density of cell nuclei as specifically stained with Hoechst was measured after 3 days to investigate the effect of lenses on cell growth (FIG. 28). The cell density did not show any significant difference between the control contact lens bearing no liposome and the contact lenses bearing 5 layers of stable liposomes after 3 days of culture. However, the cell growth was significantly lower than in control cultures with or without serum. Moreover, cultures in the presence of liposome suspensions decreased the growth of epithelial cells depending on their concentrations. However, the liposomes attached to the lenses did not impair cell growth or cell viability. In addition, the morphology of the cells (FIGS. 23 to 27) was impaired neither by control lenses nor by lenses bearing liposomes.

Indirect Biocompatibility Using an Elution Assay

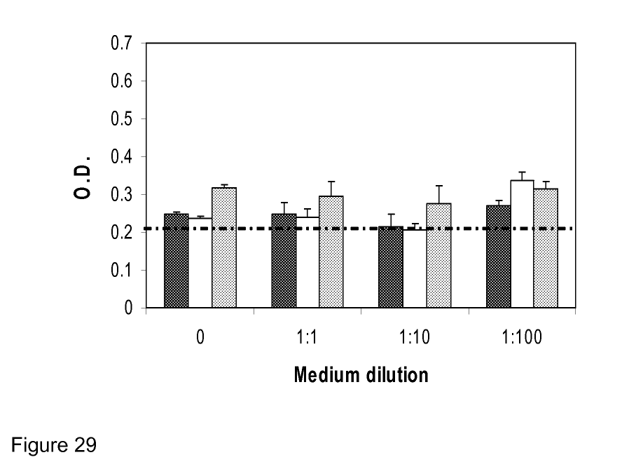

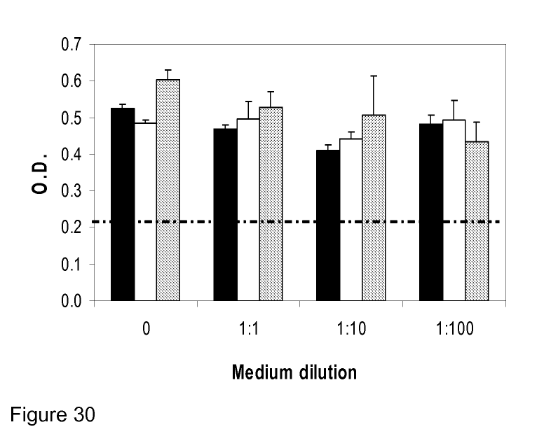

To investigate the potential cytotoxicity of products eluted from contact lenses coated with liposome layers, additional tests were conducted using the medium in which the materials have been previously soaked for 3 and 10 days. Mitochondrial activity was measured following the XTT assay. There were no significant differences between the different dilutions, and the different tested lenses and culture conditions (FIGS. 29 and 30). Therefore, this finding indicates that there is no cytotoxic compound that leaks from the lenses, even after 10 days of soaking in culture medium.

Bi-Layered Cornea

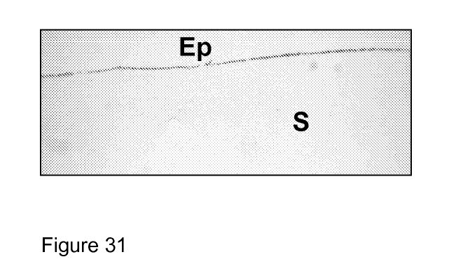

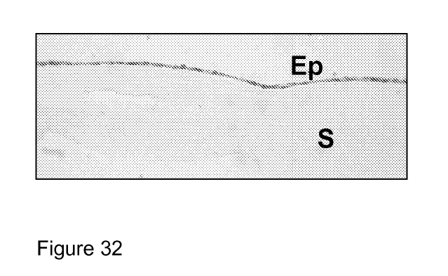

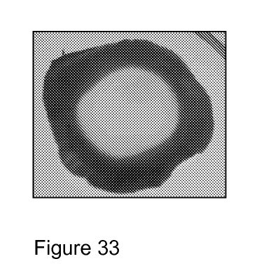

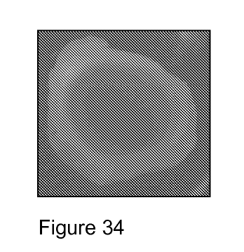

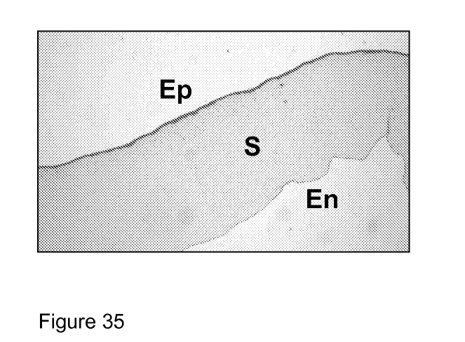

As mentioned earlier, there was no alteration of the epithelial cell layer and this was confirmed by the observation of histological sections (FIGS. 31 to 35). Cells appeared morphologically normal and formed one or two layers of cells as in the control reconstructed cornea/stroma.

Ex Vivo Testing on Rabbit Cornea

This assay confirmed that the explanted corneas that were in contact with lenses bearing liposomes were not altered. Using toluidine blue and carboxyfluorescein, there was no injury of the surface of the cornea as also seen by histological sections of the whole cornea (FIGS. 31 to 35).

Transmission Spectra of Contact Lenses Bearing a Variable Number of Liposome Layers

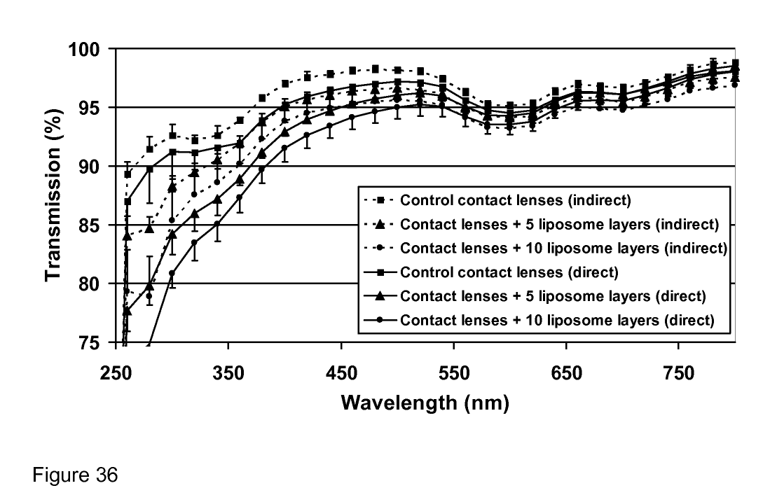

The mean indirect spectral transmittance curves of 3 tested lenses studied are shown in FIG. 36. From approximately 250 nm to 800 nm, contact lenses without liposome (control) appear to transmit more light, compared to lenses bearing layers of stable liposomes. Lenses bearing 5 layers of stable liposomes transmit more light than the ones bearing 10 layers of stable liposomes. The difference between the control lenses and the ones coated with 5 or 10 layers of liposomes was maximal at around 280 nm. Table 4 indicates that the difference in the indirect transmittance between control lenses and lenses bearing layers of stable liposomes was statistically significant at 280 nm, but was not statistically significant at 550 nm, as revealed by paired t-tests.

Table 4: Direct and Indirect Light Transmission Measurements at 280 and 550 nm for Contact Lenses (CL) with 0 (Control Lens), 5 or 10 Layers of Stable Liposomes.

| Transmission mode | | | | | | |
|---|---|---|---|---|---|---|
| Layers of liposomes on CL | Indirect | | | Direct | | |
| λ (nm) | 0 | 5 | 10 | 0 | 5 | 10 |
| 280 nm | 91.5 ± 1.1*¶ | 84.7 ± 0.9* | 78.9 ± 3.4¶ | 89.8 ± 2.9∇∂ | 79.8 ± 1.7∂§ | 74.8 ± 0.5∇§ |
| 550 nm | 97.0 ± 0.3 | 95.6 ± 0.5 | 94.8 ± 1.3 | 96.3 ± 1.2 | 95.6 ± 0.2 | 94.7 ± 0.8 |

*, ¶, ∂, ∇, §, P ≦ 0.05

FIG. 36 also shows measurements of the direct transmittance of the same contact lenses. Direct transmittance data, also summarized in Table 4, was significantly different between control lenses and lenses with liposomes. Differences between the lenses with 5 liposome layers and with 10 liposome layers were also statistically significant at 280 nm. Again, at 550 nm, there was no significant difference between any of the transmittance measured on the contact lenses for direct measurements.

Table 4 shows that at 280 nm, the direct average measurement was generally smaller than the indirect transmittance. This difference was only statistically significant for the lenses with 5 layers of liposomes. In indirect measurements diffuse light is also measured in addition to the directly transmitted light. There was no significant difference between any measurements modes for any of the contact lenses tested at 550 nm.

Conclusions

Contact lenses bearing layers of stable liposomes did not induce any significant changes in cell viability and in cell growth compared with lenses bearing no liposome. Elution assays revealed that no cytotoxic compound leaked from the lenses bearing or not liposomes. Histological analyses of reconstructed human corneas and ex vivo rabbit corneas directly exposed to liposomal lenses revealed no alteration to the cell and tissue structures.

Lenses bearing 5 or 10 layers of liposomes do not significantly affect light transmission compared to control lenses without liposomes at the wavelength of the maximal photopic sensitivity, at 550 nm. However, in the ultraviolet spectrum, direct and indirect transmittance levels of lenses with liposomes were significantly lower compared with control lenses that do not contain a UV absorber.

Although the present invention has been described hereinabove by way of specific embodiments thereof, it can be modified, without departing from the spirit and nature of the subject invention as defined in the appended claims.

What is claimed is:

1. A method of modifying a contact lens with a carrier moiety capable of associating with an agent, said method comprising contacting, in one or more steps, one or more of (a) said contact lens comprising a first ligand attached thereto and (b) said carrier moiety comprising a second ligand attached thereto, with (c) an adapter molecule capable of concurrent binding to said first and second ligands, under conditions permitting binding of said adapter molecule to said first and second ligands, wherein said first and second ligands may be the same or different.

2. The method of claim 1 comprising:
(a) incubating said contact lens comprising said first ligand attached thereto with said adapter molecule under conditions permitting binding of said first ligand to said adapter molecule; and
(b) incubating the adapter-molecule modified contact lens of (a) with said carrier moiety comprising said second ligand attached thereto under conditions permitting binding of said second ligand to said adapter molecule.

3. The method of claim 1 comprising:
(a) incubating said carrier moiety comprising said second ligand attached thereto with said adapter molecule under conditions permitting binding of said second ligand to said adapter molecule; and
(b) incubating said adapter-molecule modified carrier of (a) with said contact lens comprising said first ligand attached thereto under conditions permitting binding of said first ligand to said adapter molecule.

4. The method of claim 1 comprising incubating (i) said contact lens comprising said first ligand attached thereto with (ii) said carrier moiety comprising said second ligand attached thereto and said adapter molecule, under conditions permitting binding of said first and second ligands to said adapter molecule.

5. The method of claim 1, wherein said adapter molecule is capable of homospecific binding and said first and second ligands are the same.

6. The method of claim 1, wherein said adapter molecule is capable of heterospecific binding and said first and second ligands are different.

7. The method of claim 2 further comprising:
(c) incubating said carrier-modified contact lens of (b), wherein said carrier moiety is a first carrier moiety, with a second adaptor molecule capable of binding a ligand attached to said first carrier moiety, under conditions permitting the binding of said second adaptor molecule to said first carrier-attached ligand; and
(d) incubating said second adapter molecule-modified contact lens of (c) with a second carrier moiety which may be the same or different from said first carrier moiety, said second carrier moiety comprising a ligand attached thereto capable of binding said second adapter molecule, under conditions permitting the binding of said second carrier-attached ligand to said second adapter molecule.

8. The method of claim 7 wherein steps (c) and (d) are repeated one or more times with further adapter molecules and ligand-attached carrier moieties.

9. The method of claim 2 wherein in step (b) said carrier moiety comprising second ligands attached thereto are in the form of aggregates of carrier moieties which are formed prior to step (b) by incubating said carrier moieties with adapter molecules under conditions permitting the formation of aggregates via binding of said second ligands to said adapter molecules.

10. The method of claim 1 wherein any of said ligands are biotin and any of said adapter molecules are a biotin-binding protein.

11. The method of claim 1 wherein the carrier moieties are liposomes.

12. The method of claim 1 wherein, prior to binding said adapter molecule to said first and second ligands, said contact lens has been incubated in a solution of a polymer under conditions permitting the reaction of a first functional group with a second functional group to form a covalent link, wherein the contact lens comprises said first functional group attached thereto, the polymer comprises said second functional group attached thereto, and said first and second functional groups can react with each other to form a covalent link.

13. The method of claim 12 wherein said polymer is low-fouling.

14. The method of claim 12 wherein said solution is in cloud point conditions.

15. The method of claim 1 wherein said contact lens comprises a third functional group attached thereto, which has been activated, prior to binding of said adapter molecule to said first and second ligands, by incubating said contact lens in the presence of a coupling agent capable of reacting with said third functional group to activate it.

16. The method of claim 15 wherein said third functional group is a carboxyl or a hydroxyl group.

17. A contact lens produced by the method of claim 1.

18. A kit for modifying a contact lens comprising:
(a) a contact lens comprising a first ligand attached thereto;
(b) a carrier moiety comprising a second ligand attached thereto; and
(c) an adapter molecule capable of binding said first and second ligands.

19. The kit of claim 18 further comprising instructions setting forth the method of claim 1.

20. The kit of claim 18 further comprising a second adaptor molecule capable of binding said second ligands.

21. The kit of claim 20 further comprising instructions setting forth the method of claim 7.

22. The kit of claim 18 wherein any of said ligand is biotin and any of said adapter molecule is a biotin-binding protein.

23. The kit of claim 18 wherein said carrier moiety is a liposome.

24. A contact lens comprising carrier moieties bound thereto, wherein a first ligand is attached to said contact lens, a second ligand is attached to said carrier moieties, and said first and second ligands are concurrently bound to an adapter molecule, said first and second ligands may be the same or may be different and said adapter molecule is capable of binding said first and second ligands concurrently.

25. The contact lens of claim 24 comprising a plurality of layers of carrier moieties attached thereto.

26. The contact lens of claim 24 wherein said carrier moieties are liposomes.

27. The contact lens of claim 24 wherein, prior to binding said adapter molecule to said first and second ligands, said contact lens has been incubated in a solution of a polymer under conditions permitting the reaction of a first functional group with a second functional group to form a covalent link, wherein the contact lens comprises said first functional groups attached thereto, the polymer comprises said second functional groups attached thereto and said first and second functional groups can react with each other to form a covalent link.

28. The contact lens of claim 27 wherein said polymer is low-fouling.

29. The contact lens of claim 27 wherein the solution of polymer is in cloud point conditions.

30. The contact lens of claim 27 wherein said contact lens comprises a third functional group attached thereto, which has been activated, prior to binding said adapter molecule to said first and second ligands, by incubating said contact lens in a coupling agent capable of reacting with said third functional group to activate it.

31. The contact lens of claim 30 wherein said third functional groups are selected from hydroxyl or carboxyl groups.

32. The contact lens of claim 24 wherein the carrier moiety comprises one or more agents.

33. The contact lens of claim 32 wherein the agent is selected from the group consisting of drugs, ophthalmic drugs, proteins, hormones, enzymes, amino acids, polypeptides, medicaments, therapeutics, vitamins, antibiotics, anti-inflammatory agents and anti-rejection agents.

34. A method for administering an agent to the eye of a subject comprising placing the contact lens of claim 32 in contact with the eye of the subject.

35. A package comprising the contact lens of claim 32 together with instructions for administering an agent to or bringing an agent into contact with the eye.

36. The method of claim 34, wherein the administration comprises controlled release of the agent.

37. The method of claim 34, wherein the method is for improving wear comfort.

38. The method of claim 34, wherein the method is for treating a condition selected from ocular disease, ocular infection, ocular infectious diseases and ocular inflammation.

39. The method of claim 38, wherein the method is selected from chronic uveitis, ocular wound, dry eye, chronic keratitis, glaucoma or corneal ulcer.

40. The method of claim 34, wherein the method is for preventing the rejection of a corneal graft.

41. The contact lens of claim 32 wherein the agent is a sensor.

42. A method of sensing an ocular analyte in an eye of a subject, comprising placing the contact lens of claim 41 in contact with the eye of the subject and assessing the analyte in accordance with the signal derived from the sensor.

43. A method for modifying a contact lens which comprises a functional group attached thereto, comprising incubating said contact lens in the presence of a coupling agent capable of reacting with said functional group to activate said functional group.

44. The method of claim 43 wherein said third functional groups are hydroxyl or carboxyl groups.

45. A contact lens produced by the method of claim 43.

46. A method of modifying a surface comprising contacting said surface with a solution of a polymer in cloud point conditions under conditions permitting the reaction of a first functional group with a second functional group, wherein said surface comprises said first functional group attached thereto, said polymer comprises said second functional group attached thereto and said first and second functional groups can react with each other to form a covalent link.

47. The method of claim 46 wherein said surface is a surface of a contact lens.

48. The method of claim 46 wherein said polymer is low-fouling.

49. A surface produced by the method of claim 46.

50. A kit for modifying a surface comprising first functional groups attached thereto, said kit comprising a solution of a polymer in cloud point conditions and instructions for incubating said surface in said polymer solution, wherein said polymer comprises second functional groups attached thereto and said first and second functional groups can react with each other to form a covalent link.

51. The kit of claim 50 wherein said polymer is low-fouling.

52. An article of manufacture comprising a surface onto which a polymer has been covalently linked by incubating said article of manufacture or surface thereof in a solution of the polymer in cloud point conditions under conditions permitting the reaction of first and second functional groups, wherein said surface comprises said first functional groups attached thereto, said polymer comprises said second functional groups attached thereto and said first and second functional groups can react with each other to form a covalent link.

53. The article of manufacture of claim 52 wherein said article is a contact lens and wherein said surface is a surface of a contact lens.

54. The article of manufacture of claim 52 wherein said polymer is low-fouling.
